(19) United States
(12) Patent Application Publication
(10) Pub. No.: US 2025/0090606 A1
(43) Pub. Date: Mar. 20, 2025

(54) COMPOSITIONS FOR TREATMENT OF A FATTY LIVER OR FIBROTIC LIVER DISEASE

(71) Applicant: NORDIC REBALANCE A/S, Hillerød (DK)

(72) Inventors: Hans ISRAELSEN, Allerød (DK); Mads Egerod ISRAELSEN, Copenhagen Ø (DK)

(21) Appl. No.: 18/727,324

(22) PCT Filed: Jan. 13, 2023
(86) PCT No.: PCT/EP2023/050782

§ 371 Date: Jul. 8, 2024

(30) Foreign Application Priority Data
Jan. 14, 2022 (DK) PA202200037

(51) Int. Cl. A61K 35/747; A23L 7/10; A23L 7/104; A61K 35/00; A61K 36/899; A61P 1/16

(52) U.S. Cl. CPC A61K 35/747; A23L 7/104; A23L 7/198; A61K 36/899; A61P 1/16; A23V 2400/169; A61K 2035/115; A61K 2236/19

(57) ABSTRACT

The present invention is directed to fermented cereal compositions for treatment of a fatty liver disease or a fibrotic liver disease.

### FIELD OF THE INVENTION

The present invention relates to the treatment of a fatty liver disease or a fibrotic liver disease, including a fibrotic liver disease associated with gut-dysbiosis, such as alcoholic liver disease (ALD) or non-alcoholic fatty liver disease (NAFLD). The invention is directed to fermented cereals and probiotics, preferably anti-inflammatory microorganisms, as treatment effectors.

### BACKGROUND OF THE INVENTION

Liver fibrosis can be defined as an excessive accumulation in the liver of extracellular matrix proteins, including collagen. Advanced liver fibrosis may result in cirrhosis, portal hypertension, liver cancer and ultimately liver failure, and liver transplantation will often be the only option to consider when seeking to treat advanced cases of liver fibrosis.

Emerging anti-fibrotic therapies are aimed at inhibiting the accumulation of fibrogenic cells and/or preventing the deposition of extracellular matrix proteins. Although many such therapeutic interventions are effective in experimental models of liver fibrosis, their efficacy and safety in humans is largely unknown.

There exists a need for safe and effective anti-fibrotic therapies aimed at inhibiting the accumulation of fibrogenic cells and/or preventing the deposition of extracellular matrix proteins in human beings.

WO 2007/036230 describes compositions comprising fermented cereal gruel for treatment of gastrointestinal diseases, including IBS and IBD.

Krag et al. (2012) describes that fermented cereal compositions may induce remission in ulcerative colitis (World J. Gastroenterol., vol. 18(15), pp. 1773-1780).

Krag et al. (2013) describes that fermented cereal compositions are efficacious in patients with active ulcerative colitis (Inflamm. Bowel Dis., vol. 19, pp. 2584-2592).

Bednarska et al. (2022) describes that fermented oat gruel may have a beneficial effect on the colonic mucosal barrier in patients with irritable bowel syndrome (Frontiers in Nutrition, Dec. 8, 2022 (online publ.)).

Hansen et al. (2022) describes an effect of a fermented cereal product on hepatic stellate cell activity in patients with advanced alcohol-related liver disease (Hepatology, vol. 76 (Suppl. 1), S982-S983).

### SUMMARY OF THE INVENTION

The present invention in a first aspect relates to a treatment of a fatty liver disease or a fibrotic liver disease, including a fibrotic liver disease associated with gut-dysbiosis. There is also provided fermented cereal compositions for use in a treatment of a fatty liver disease or a fibrotic liver disease, including ready-to-use compositions and daily dosage forms comprising a fermented cereal composition.

The fermented cereal compositions are obtained by fermenting a cereal gruel with Lactobacillus plantarum cells, preferably L. plantarum 299 and/or L. plantarum 299v. The fermented cereal composition may be dried or heat-treated once the fermentation has been completed.

Examples of fibrotic liver diseases include alcoholic liver disease (ALD) or non-alcoholic fatty liver disease (NAFLD).

Fibrotic liver disease is a potentially fatal disease, and the end stage of fibrotic liver disease, termed cirrhosis, is associated with high mortality.

The progression of liver disease from fatty liver disease via liver fibrosis to cirrhosis is not characterized by specific symptoms, and the disease process is usually diagnosed only at advanced stages of the disease.

However, certain diagnostic methods aim to improve a diagnosis of a fibrotic liver disease at the earlier stages of the disease.

As there are no disease-specific symptoms for a fibrotic liver disease, medical practitioners typically monitor the progression of a fibrotic liver disease by performing laboratory tests, by diagnostic imaging, and by direct investigation of tissue samples.

In one aspect, the present invention is directed to a combination of an efficient amount of a fermented cereal composition and an efficient amount of probiotic microorganisms, and optionally also phospholipids, for use in the treatment of a fatty or fibrotic liver disease. The efficient amount of the fermented cereal composition may be administered to an individual, such as a human being, in a ready-to-use formulation, such as a daily dosage form. A daily dosage form may be administered several times daily as two or more sub-dosage forms that collectively constitute a daily dosage form.

The cereal is preferably oat gruel, and the oat gruel is preferably fermented by the probiotic microorganisms, preferably L. plantarum 299 and/or L. plantarum 299v. Additional probiotic microorganisms may be added after the fermentation, including non-pathogenic lactic acid bacteria, such as, but not limited to, bacteria of the genera Lactobacillus, Lactococcus and Bifidobacterium.

The pH of the fermentation mixture is preferably below pH 5.5 after fermentation for about 12 to 24 hours.

The present invention is also directed to dosage forms suitable for a daily intake, including daily dosage forms of effective dosages of a cereal gruel having been fermented by probiotic microorganisms, including non-pathogenic lactic acid bacteria, such as, but not limited to, bacteria of the genera Lactobacillus, Lactococcus and Bifidobacterium. Preferably, the probiotic microorganisms are L. plantarum 299 and/or L. plantarum 299v.

There is also provided methods of treatment involving a daily intake of effective dosages of a cereal gruel having been fermented by probiotic microorganisms, including non-pathogenic lactic acid bacteria, such as, but not limited to, bacteria of the genera Lactobacillus, Lactococcus and Bifidobacterium. Preferably, the probiotic microorganisms are L. plantarum 299 and/or L. plantarum 299v.

### Definitions

A “probiotic microorganism” is generally regarded as safe (GRAS) for human consumption and capable of inhabiting the gastrointestinal tract of an individual, including a human being. As the environment and microbiota of the gastrointestinal tract is different in different human beings, the metabolism of probiotic microorganisms is also different in different human beings. The metabolism of probiotic microorganisms is determined by many diverse factors, such as, e.g., the gut microbiota (i.e. the gut bacterial habitat), the general “health” status of the gut, and the diet of a human being.

The term “postbiotics” include fermentation products generated by probiotic microorganisms during fermentation of a substrate, such as a cereal gruel. As the metabolism of probiotic microorganisms is different in different human beings (cf. “probiotics” above), different kinds, or different amounts, of “postbiotics” will be produced by probiotic microorganisms in different human beings under different sets of circumstances.

A probiotic microorganism capable of metabolism and/or cell division when inhabiting the gastrointestinal tract of an individual is deemed to be “viable”. Viability of microbial organisms may be confirmed by a determination of “colony forming units” (“cfu”) in a suitable growth medium.

“Non-viable” probiotic microorganisms are unable to generate “colony forming units”. Viable probiotic microorganisms may become “non-viable” by any treatment that disrupts metabolism and results in irreversible cellular damage. Certain types of “non-viable” probiotic microbial cells with a largely intact macro-cellular structure are termed “ghost cells”.

Probiotic microbial cell components are components originating from viable or non-viable probiotic microorganisms.

A “cereal” as used herein is defined as any grain or seed produced by a plant from the grass family. Commonly available grains are barley, corn, millet, oats, quinoa, rice, rye, sorghum, triticale, wheat, rice and cassava. Oat is one preferred grain.

A “cereal gruel” as used herein is defined as cereal grains, flakes, meals, extracts or flour having been suspended in—and preferably boiled in—an aqueous liquid such as water. Compositions containing enzymes and/or appropriate amounts of carbon and/or energy sources may be added to the cereal gruel to facilitate a subsequent microbial fermentation and/or to provide a desired rheology of the fermented product. Cereal gruel is used herein to cover any rheological form of a suspended and boiled cereal.

A “fermented cereal composition” is a product resulting from fermenting cereal gruel with one or more non-pathogenic microorganisms. The fermented cereal gruel may contain one or more probiotic microorganisms, including non-pathogenic lactic acid bacteria, and the microorganisms may be dead or alive. Fermented cereal compositions according to the present invention are also referred to herein as a “fermented product” or “fermented oat gruel”.

The term “cereal-fermenting probiotic microorganism” includes a non-pathogenic lactic acid bacteria capable of fermenting a cereal gruel and generating a fermentation mixture having a pH of less than 5.5 in from preferably 12 to 24 hours.

An “efficient amount” is an amount of a fermented cereal composition having been obtained by fermentation of a cereal gruel with probiotic microorganisms, wherein the fermented cereal composition is administered daily, or at least several times weekly, to a patient in need thereof, and results over time in a reduction of disease symptoms, if any, or a normalization of disease markers of the patient being treated.

A “treatment effecter” includes a fermented cereal composition, or a part thereof, that exerts an effect and reduces one or more disease symptoms, if any, or a normalization of disease markers associated with a fatty or fibrotic liver disease in an individual.

“Gut dysbiosis” is defined as a harmful imbalance in the composition and/or the metabolism of the microorganisms colonizing the digestive tract of an individual, including a human being. The harmful imbalance may lead to disease development and/or progression.

### BRIEF DESCRIPTION OF THE FIGURES

FIG. 1 illustrates the progression of liver damage in fatty liver disease, liver fibrosis and cirrhosis. In fatty liver disease, deposits of fat lead to liver enlargement. In liver fibrosis, scar tissue forms, and in cirrhosis, growth of connective tissue destroys/displaces liver cells.

FIG. 2 is a Waterfall plot showing the percentage change α-smooth muscle actin (α-SMA) expression after six month treatment with Referm or Fresubin.

FIG. 3 illustrates effects on various markers, incl. alpha-ASM (ASMA), LSM (TE), Shear wave elastography (SWE), Enhanced Liver Fibrosis (ELF) and Pro-C3 collagen marker (PROC3).

FIG. 4 is a Table illustrating results in the form of changes from baseline to end of study for the markers in FIG. 3.

FIG. 5 is am illustration of a pathophysiological overview of ALD and NAFLD (from Albillos 2020, JHEP).

### DETAILED DESCRIPTION OF THE INVENTION

Preferred embodiments of the present invention are described herein below.

### Probiotic and Postbiotic Effects of Lactobacillus Plantarum Strains 299v and 299

Colonization and/or growth in the gut of an individual, including a human being, of L. plantarum cells of strains 299v and 299 exert probiotic effects. However, the metabolism of probiotic L. plantarum 299v and 299 is affected differently by different gut environmental conditions, including differences in the gut microbiota. For this reason, growth and metabolism of L. plantarum 299v and 299 will differ from individual to individual.

Differences in gut environmental conditions and microbiota will result in production of different amounts of different metabolites, including postbiotics, by L. plantarum strains 299v and 299 in different individuals, including human beings.

Production by L. plantarum 299v and 299 of different postbiotics in different individuals is likely to result in different effects of a treatment over time, and such different effects may complicate the management of a treatment of a gut-related disease, such as a fatty liver disease or a fibrotic liver disease. Hence, there is a need for aligning treatment protocols and the medical management of different gastrointestinal diseases, including liver diseases, such as fatty liver disease or a fibrotic liver disease, in an individual, including a human being.

In an attempt to align treatment protocols and disease management conducted by medical professionals, different types of compositions comprising cereal gruel fermented by L. plantarum strains 299v and/or 299 for use in treatment of liver diseases are provided in accordance with the present invention.

The fermented cereal compositions for use in treatment of a liver disease that are obtained by fermenting cereal gruel, preferably oat gruel, with one or both of L. plantarum strains 299v and 299 may, e.g., be formulated as a ready-to-use daily dosage form, including a fermented cereal composition in any edible or drinkable, liquid or solid form suitable for daily administration to an individual suffering from a liver disease.

In one embodiment, there is provided a ready-to-use fermented cereal composition comprising at least 10 gram (g) (dry weight) of fermented cereal obtained by fermenting a cereal gruel with Lactobacillus plantarum cells selected from Lactobacillus plantarum 299 and Lactobacillus plantarum 299v for use in a treatment of a patient suffering from a liver disease, including a fatty liver disease and a fibrotic liver disease. The treatment preferably comprises a daily administration of the ready-to-use composition to the patient.

The amount of fermented cereal and the number of colony forming units (cfu) of Lactobacillus plantarum 299 and Lactobacillus plantarum 299v in the composition may be independently regulated according to the probiotic and/or postbiotic needs of the patient.

Accordingly, when there is a need for administration of higher amounts of fermented cereal composition, a fermented cereal composition comprising at least 12 gram (g) (dry weight) of cereal, such as at least 14 gram (g) (dry weight) of cereal, for example at least 16 gram (g) (dry weight) of cereal, such as at least 18 gram (g) (dry weight) of cereal, for example at least 20 gram (g) (dry weight) of cereal, such as at least 22 gram (g) (dry weight) of cereal, for example at least 24 gram (g) (dry weight) of cereal, such as at least 26 gram (g) (dry weight) of cereal, for example at least 28 gram (g) (dry weight) of cereal, such as at least 30 gram (g) (dry weight) of cereal, for example at least 32 gram (g) (dry weight) of cereal, such as at least 34 gram (g) (dry weight) of cereal, for example at least 36 gram (g) (dry weight) of cereal, such as at least 38 gram (g) (dry weight) of cereal, for example at least 40 gram (g) (dry weight) of cereal, such as at least 60 gram (g) (dry weight) of cereal, for example at least 80 gram (g) (dry weight) of cereal, such as at least 100 gram (g) (dry weight) of cereal may be administered on a daily basis to an individual suffering from a liver disease.

The fermented cereal composition is preferably obtained by fermenting a cereal gruel with Lactobacillus plantarum 299 and/or Lactobacillus plantarum 299v.

Independently of the above-stated amounts of fermented cereal, and with a view to balancing the probiotic and/or postbiotic needs of an individual suffering from a liver disease, the fermented cereal composition may comprise any number of colony forming units (cfu) of Lactobacillus plantarum 299 and/or Lactobacillus plantarum 299v, including no colony forming units (cfu) of Lactobacillus plantarum 299 and/or Lactobacillus plantarum 299v.

Accordingly, for patients in need of boosting probiotic effects in the gut, such as, e.g., boosting short chain fatty acid (SCFA) synthesis, Lactobacillus plantarum 299 and/or Lactobacillus plantarum 299v synthesis in the gut of short chain fatty acids would be desirable, and such a boosting effect would be achieved, e.g., by administration of a higher number of colony forming units (cfu) of Lactobacillus plantarum 299 and/or Lactobacillus plantarum 299v to an individual, including a human being.

Higher numbers of colony forming units (cfu) of Lactobacillus plantarum 299 and/or Lactobacillus plantarum 299v in the ready-to-use product suitable for daily administration to an individual suffering from a liver disease would be, e.g., at least 10⁸ cfu Lactobacillus plantarum 299 and/or Lactobacillus plantarum 299v, but the number could be higher based on patient needs, such as, e.g., at least 10⁹ or 10¹⁰ cfu Lactobacillus plantarum 299 and/or Lactobacillus plantarum 299v.

Lower numbers of colony forming units (cfu) of Lactobacillus plantarum 299 and/or Lactobacillus plantarum 299v in the ready-to-use product suitable for daily administration to an individual suffering from a liver disease would be required for individuals diagnosed with no need or a reduced need for probiotic boosting of the gut, such as SCFA synthesis by probiotic Lactobacillus plantarum 299 and/or Lactobacillus plantarum 299v.

Lower numbers of colony forming units (cfu) of Lactobacillus plantarum 299 and/or Lactobacillus plantarum 299v in the ready-to-use product suitable for daily administration to an individual suffering from a liver disease would be, e.g., from about 10⁷ to about 10⁸ cfu Lactobacillus plantarum 299 and/or Lactobacillus plantarum 299v, such as from about 10⁶ to about 10⁷ cfu Lactobacillus plantarum 299 and/or Lactobacillus plantarum 299v, for example from about 10⁵ to about 10⁶ cfu Lactobacillus plantarum 299 and/or Lactobacillus plantarum 299v, such as from about 10⁴ to about 10⁵ cfu Lactobacillus plantarum 299 and/or Lactobacillus plantarum 299v, for example from about 10³ to about 10⁴ cfu Lactobacillus plantarum 299 and/or Lactobacillus plantarum 299v, such as from about 10² to about 10³ cfu Lactobacillus plantarum 299 and/or Lactobacillus plantarum 299v,

However, the cfu number could be even lower based on patient needs as evaluated by medical practitioners, such as, e.g., from about 10 to about 500 cfu Lactobacillus plantarum 299 and/or Lactobacillus plantarum 299v. In one embodiment, the ready-to-use product suitable for daily administration to an individual suffering from a liver disease contains no viable Lactobacillus plantarum 299 and/or Lactobacillus plantarum 299v.

There are different ways of regulating the cfu number in the ready-to-use product suitable for daily administration to an individual suffering from a liver disease. For example, once the Lactobacillus plantarum 299 and/or Lactobacillus plantarum 299v fermentation of the cereal gruel is complete, as evaluated, e.g., by the pH of the cereal gruel, which should preferably be lower than 5.5 when the fermentation is terminated, it is possible to subject the fermented cereal composition to a gentle heat treatment sufficient to inactivate some, but not all, Lactobacillus plantarum 299 and/or Lactobacillus plantarum 299v cells.

The reduction in cfu numbers will depend on the temperature and the duration of the heat inactivation of Lactobacillus plantarum 299 and/or Lactobacillus plantarum 299v cells. A skilled practitioner may use state-of-the-art cell counting devices and methodologies to readily determine the cfu number in a fermented cereal during a heat treatment, and standard curves estimating Lactobacillus plantarum 299 and/or Lactobacillus plantarum 299v survival rates at different temperatures and time points during a heat inactivation step may be generated.

Reduced Lactobacillus plantarum 299 and/or Lactobacillus plantarum 299v cfu numbers may also be obtained by drying the fermented cereal composition. Various state-of-the-art drying methods are available to the skilled practitioner, including, e.g., spray-drying and freeze-drying. Drying the fermented cereal composition will generate a dried, ready-to-use composition suitable for daily administration. The dried composition may be mixed with ingredients of a daily diet or resuspended to generate a drinkable ready-to-use composition suitable for oral administration to an individual suffering from a liver disease.

In one embodiment, an aqueous solution optionally comprising one or more postbiotics, such as, e.g., short chain fatty acids, is added to the dried fermented cereal composition prior to formulating an edible ready-to-use composition suitable for daily administration. The addition of the aqueous solution optionally comprising one or more postbiotics, including short chain fatty acids, may take place in an agglomeration step in order to generate a dried and agglomerated ready-to-use composition suitable for daily administration. Agglomeration of dried compositions is a methodology that is well-known to the skilled person, and agglomeration steps are often used for improving liquid resuspension or solubilization of dried compositions.

A dried and agglomerated ready-to-use composition would be used as an ingredient in a daily diet or resuspended in a suitable amount of liquid prior to daily administration to an individual suffering from a liver disease.

Apart from providing a means to regulating cfu numbers and viability of Lactobacillus plantarum 299 and/or Lactobacillus plantarum 299v cells, there are additional advantages associated with formulating the ready-to-use composition for daily administration as a dried or agglomerated composition. For example, the shelf-life of a dried or agglomerated ready-to-use formulation comprising fermented cereal will increase compared to the shelf-life of a liquid ready-to-use formulation.

Additionally, it is possible to readily generate in accordance with the present invention a cfu standardized ready-to-use composition for daily administration to an individual, wherein the composition has a shelf-life of more than, e.g., 3 months, such as more than 6 months, for example more than 9 months, such as more than 12 months. The longer shelf-life would be achieved, e.g., by adding a freeze-dried culture with a known cfu amount of Lactobacillus plantarum 299 and/or Lactobacillus plantarum 299v cells to the dried or agglomerated ready-to-use composition for daily administration. In this way, the dried or agglomerated ready-to-use composition would be suitable for administration on a daily basis to individuals having a need for a probiotic boost to the gut microbiota.

Increased cfu numbers of Lactobacillus plantarum 299 and/or Lactobacillus plantarum 299v in the ready-to-use composition for daily administration may also be obtained, e.g., by prolonging the fermentation by, e.g., about 6 to 12 hours, and/or by increasing the amount of fermented cereal composition in the ready-to-use product suitable for daily administration to an individual suffering from a liver disease.

Irrespective of the above-described probiotic effects associated with ready-to-use fermented cereal compositions containing viable Lactobacillus plantarum 299 and/or Lactobacillus plantarum 299v cells, ready-to-use dosage forms suitable for daily administration to an individual suffering from a liver disease according to the present invention have been shown to also exert a postbiotic effect in the gut of an individual. This is clear from a daily administration of ready-to-use compositions having no or very low numbers of viable Lactobacillus plantarum 299 and/or Lactobacillus plantarum 299v cells.

It is surprising and highly unexpected that ready-to-use fermented cereal compositions and dosage forms suitable for daily administration to an individual suffering from a liver disease may contain no or very few viable Lactobacillus plantarum 299 and/or Lactobacillus plantarum 299v cells. An exclusively postbiotic effect associated with a cereal gruel fermented with Lactobacillus plantarum 299 and/or Lactobacillus plantarum 299v cells has not been described in the prior art.

### Liver Diseases

The present invention makes it possible to treat liver diseases, including fatty liver diseases and fibrotic liver diseases, by administering a fermented cereal composition to an individual in need thereof.

According to presently preferred hypotheses, probiotic and/or postbiotic effects in the gut exerted by the claimed fermented cereal compositions prevent liver disease progression and liver disease development, initiate regression of a liver disease, or postpone progression of a liver disease, including a fatty liver disease or a fibrotic liver disease.

The present invention also aims to treat gut dysbiosis with the aim of preventing liver disease progression and liver disease development, initiating regression of a liver disease, or postponing the progression of a liver disease, including a fatty liver disease or a fibrotic liver disease.

A fibrotic liver disease is characterized by the accumulation of fibrosis in the liver. Research indicates that gut dysbiosis may play an important role in the pathogenesis in alcoholic liver disease (ALD)¹, non-alcoholic liver disease (NAFLD)², some types of cholestatic liver disease (CLD) and idiosyncratic drug induced liver disease (DILI)³.

Accordingly, in one embodiment, the fibrotic liver diseases described herein above are associated with gut dysbiosis, and gut dysbiosis may lead to gut leakiness and subsequent translocation of bacteria and macromolecules from the gut to the liver. This translocation may initiate inflammation in the liver, and liver inflammation is believed to be a cause of generation of fibrotic tissue in the liver⁴. This interaction between the gut and the liver associated with disease processes is referred to as the gut-liver axis.

The natural history of these liver diseases is progressive and traditionally divided in following stages. The milder liver diseases of fat accumulation in the liver (steatosis), and the more harmful stages including inflammation (steatohepatitis) leading to fibrosis (scar tissue), cirrhosis (severe fibrosis and decreased liver function) and liver cancer.

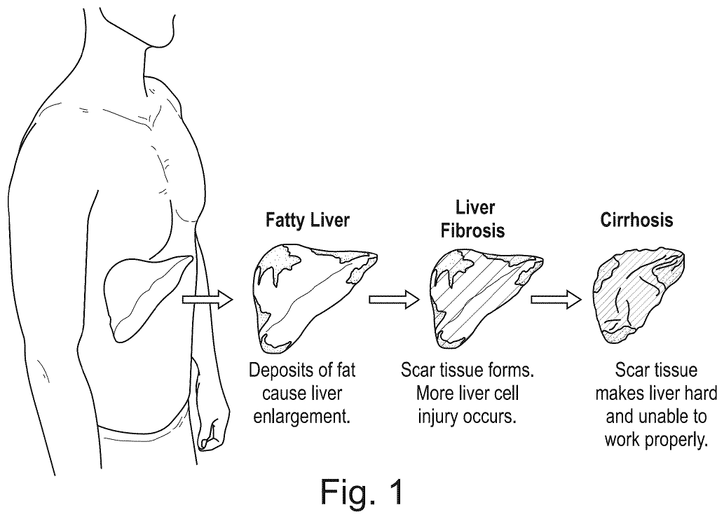

The severity of the liver disease is correlated to the accumulation of fibrotic tissue in the liver. Accumulation of fibrotic tissue in liver disease is illustrated in FIG. 1.

The following blood markers, assays, scores and imaging methods can be used for identifying and monitoring the progression of a liver disease: Bilirubin, albumin, coagulation factors, international normalized ratio, prothrombin time, mean corpuscular volume (MCV), aspartate aminotransferases (AST), alanine aminotransferases (ALT), alkaline phosphatases, immunoglobuline-A (IgA), gammaglutamyltranspeptidases (GGT), glutamic oxaloacetic transaminase (GOT), glutamic pyruvic transaminase (GPT), ferritin, platelet count, Enhanced Liver Fibrosis (ELF) test and elastography (Transient elastography and 2D Shear wave elastography).

Liver diseases can also be categorized based on etiology (the most likely cause of liver injury).

Exemplary liver diseases falling within the definition of a fatty liver or fibrotic liver disease used herein are introduced herein below.

### Alcoholic Liver Diseases

Alcohol overuse is one of the main risk factors and etiological factors leading to liver disease. Alcoholic liver disease (ALD) is the umbrella term covering all stages of liver disease that is considered to be related to alcohol overuse. ALD includes alcoholic steatosis, alcoholic steatohepatitis, alcoholic liver fibrosis and alcoholic cirrhosis.

According to international guidelines the diagnosis of alcoholic liver disease (ALD) is based on a combination of features, including a history of significant alcohol intake (excess alcohol consumption>30 gram/day), clinical evidence of liver disease, and supporting laboratory abnormalities^{5,6}. Laboratory tests should be done to exclude other etiologies and to confirm the diagnosis.

### Alcoholic Fatty Liver

Alcoholic fatty liver is an early and reversible consequence of excessive alcohol consumption. Fatty liver develops in the majority of individuals who consumes more than 60 g of alcohol per day, but may also occurs in people who drink less⁶.

In some individuals alcohol consumption over a longer period leads to inflammation and alcoholic liver fibrosis. Appeal to alcohol reduction is currently the only effective intervention.

### Alcoholic Liver Fibrosis and Compensated Cirrhosis

Alcoholic liver fibrosis (ALF) is the precursor of alcoholic cirrhosis. ALF and compensated cirrhosis rarely causes specific symptoms. It is suspected in individuals having a history of alcohol overuse and laboratory abnormalities suggesting liver ALD.

The diagnosis of alcoholic liver fibrosis and compensated cirrhosis are traditionally done by liver biopsy. The cut-off for significant ALF is Ishak Score 3-5 and Metavir F2-3, and the cut-off for cirrhosis is Ishak score 6 and Metavir F4. However, new non-invasive methods for diagnosing and grading liver fibrosis have been validated, but no consensus has been reached as to which methods to use in the clinic.

These measurements include imaging by shear wave and elastography⁷ and a panel of indirect serological makers used in algorithms to calculate the probability of liver fibrosis (Aspartate aminotransferases to platelet ratio index, FibroTest®, Fibrometer A®, Hepascore®)⁵.

The specific cut-off value has been correlated and therefore transmittable to fibrosis stages and cirrhosis due to the Ishak Score and Metavir. There is no treatment available for ALF and alcoholic cirrhosis except for appeal to alcohol reduction.

### Alcoholic Steatohepatitis

Alcoholic steatohepatitis (ASH) is a clinical syndrome, i.e. recent onset of jaundice and/or ascites in a patient with ongoing alcohol overuse or recently terminated alcohol overuse⁵. Mild ASH is frequently observed in people with ALF without causing any symptoms, however severe ASH has a 90-days mortality of more than 30%.

The largest randomized controlled trail⁸ on severe ASH used following definition: A clinical diagnosis of alcoholic hepatitis, an average alcohol consumption of more than 80 g per day for men and more than 60 g per day for women, a serum bilirubin level greater than 80 μmol per liter and a Maddrey discriminant function of 32 or higher.

Exclusion criteria in the above-described trial were: jaundice for more than 3 months, cessation of alcohol consumption for more than 2 months, the presence of other causes of liver disease, a serum aspartate aminotransferase level greater than 500 IU per liter or serum alanine transaminase level greater than 300 IU per liter. First-line therapy in patients with severe ASH includes corticosteroids or, in case of ongoing sepsis, pentoxifylline⁵. N-acetylcysteine may be useful in patients with severe ASH receiving corticosteroids⁵.

### Non-Alcoholic Fatty Liver Disease

Non-alcoholic fatty liver disease (NAFLD) is—like alcoholic fatty liver disease (ALD)—a generic term covering non-alcoholic fatty liver (NAFL), and non-alcoholic steatohepatitis (NASH) characterized by inflammation and fibrosis which may progress to cirrhosis, liver failure, and liver cancer⁹.

NAFLD is associated with type 2 diabetes mellitus, dyslipidemia, obstructive sleep apnea, obesity and metabolic syndrome. The links between these diseases/disorders are not well understood.

To diagnose NAFLD it is required that (a) there is evidence of hepatic steatosis, either by imaging or by histology and (b) there are no causes for secondary hepatic fat accumulation such as significant alcohol consumption, use of steatogenic medication or hereditary disorders⁹.

NAFLD is considered as an exclusion diagnosis in where other cause of liver injury should be excluded: Excessive alcohol consumption, hepatitis C (genotype 3), Wilson's disease, lipodystrophy, starvation, parenteral nutrition, abetalipoproteinemia, medications (e.g., amiodarone, methotrexate, tamoxifen, corticosteroids), microvesicular steatosis, Reye's syndrome, acute fatty liver of pregnancy, HELLP syndrome, inborn errors of metabolism⁹.

Management of NAFLD includes lifestyle intervention such as weight loss, exercise and reduced alcohol use.

### Non-Alcoholic Fatty Liver

Non-alcoholic fatty liver (NAFL) is regarded as a benign condition like obesity. Both of these conditions have a close relationship and predispose to cardiovascular diseases and diabetes mellitus. NAFL is regarded as a precursor to non-alcoholic steatohepatitis.

### Non-Alcoholic Fatty Liver Disease With Steatohepatitis and/or Liver Fibrosis

The presence of metabolic syndrome is a strong predictor for the presence of steatohepatitis in patients with NAFLD and may be used to best identify patients with persistently abnormal liver biochemistries⁹.

Non-alcoholic steatohepatitis (NASH) is like alcoholic liver fibrosis traditionally diagnosed by liver biopsy, however non-invasive methods to identify advanced fibrosis in patients with NAFLD include the NAFLD Fibrosis Score, Enhanced Liver Fibrosis (ELF®) panel and transient elastography.

There is no broad consensus on treatment of NASH, but the American Association of Studying the Liver recommends Pioglitazone and Vitamin E (a-tocopherol) to treat steatohepatitis in patients with biopsy-proven NASH⁹.

### Cholestatic Liver Disease

Cholestasis is an impairment of bile formation and/or bile flow which may be clinically present with fatigue, pruritus and, in its most overt form, jaundice. Cholestatic liver disease covers a number of liver disease including primary biliary cholangitis/cirrhosis, primary sclerosing cholangitis and IgG4-associated cholangitis¹⁰.

What initiate these diseases is far from well understood, but gut bacteria may play an important role in the activation of the immune system. Treatment of these diseases includes use of ursodeoxycholic acid. Liver transplantation is strongly considered in patients with advanced disease.

### Primary Biliary Cholangitis/Cirrhosis

Diagnosis of Primary biliary cholangitis/cirrhosis (PBC) is made on a combination of abnormal serum alkaline phospathase (derived from the liver) for at least 6 months and presence of antimitochondrial antibodies.

### Primary Sclerosing Cholangitis

Primary sclerosing cholangitis (PSC) is a chronic, cholestatic liver disease that is characterized by an inflammatory and fibrotic process affecting both intra and extrahepatic bile ducts.

A diagnosis of PSC is made when elevated serum markers of cholestasis alkaline phosphatase and gamme-glutamyltranspeptidase and magnetic resonance cholangiopancreatography or endoscopic cholangiopancreatography show characteristic bile duct changes with multifocal strictures and segmental dilatations.

Other causes of secondary sclerosing cholangitis and other cholestatic disorders should be excluded.

### Decompensated Cirrhosis and Portal Hypertension

In further aspects of the present invention, cirrhosis in an individual can also be treated by administration of the treatment effectors pertaining to the present invention.

Cirrhosis is the stage of a liver disease characterized by severe fibrosis and decreased liver function.

Several complications occur due to the cirrhosis including esophageal variceal bleeding, hepatic encephalopathy, ascites, spontaneous bacterial peritonitis and renal failure. Decompensated cirrhosis is defined as when one of these complications occurs.

Decompensated cirrhosis is associated with gut dysbiosis and gut leakiness which lead to translocation of bacteria and macromolecules from the gut to the liver, but gut dysbiosis and gut leakiness may also be one of the mechanisms leading from compensated cirrhosis to portal hypertension and decompensation.

### Portal Hypertension and Translocation of Gut Bacteria and Macromolecule

The blood from the gut is lead to the liver via the portal vein. Portal hypertension is defined as elevation of hepatic venous pressure gradient to >5 mmHg. Portal hypertension and the severity thereof is a predictor of the following complications to cirrhosis.

### Hepatic Encephalopathy

Due to the West Haven Criteria altered mental status and generalized motor disturbance are the two major components of hepatic encephalopathy (HE)¹². The cause it is categorized in type A—acute liver failure; type B—bypassing the liver without liver disease; type C—cirrhosis and portal hypertension. Type C is subdivided by the severity into covert (minimal and grade 1) and ouvert (grade 2-4). It is widely accepted that toxins arisen from gut causes the HE.

### Ascites and Spontaneous Bacterial Peritonitis

Ascites can be caused by cirrhosis and portal hypertension. Spontaneous bacterial peritonitis (SBP) is thought to be a consequence of bacterial translocation from the gut to the ascites fluid. SBP is defined as a neutrophil cell count of 250×10⁶/L in the ascites fluid with no other reason (e.g. gut perforation) as the likely cause. Antibiotics are used in the treatment and secondary prophylaxis of SBP.

### Esophageal Variceal Hemorrhage

Esophageal varices develop in patients with cirrhosis and diagnose is done by endoscopic visualization. The grade of the varices and the risk of bleeding are closely related to the severity of the portal hypertension. Secondary prophylaxis of esophageal variceal hemorrhage includes per oral administration of antibiotics, which is thought to eradicate or reduce the amount of gram negative bacteria in the gut.

### Drug Induced Liver Injury

Although drug induced liver injury (DILI) may not fall within the categories of a fatty liver disease or a fibrotic liver disease, drug induced liver injury (DILI) shall never-the-less be considered a disease treatable in accordance with the present invention.

DILI is a broad spectrum of diseases defined as any injury to the liver by a prescribed medication, over the counter medication, herb, or dietary supplement. Intrinsic and “idiosyncratic” DILI are commonly thought to arise by different pathophysiologic mechanisms.

This patent only concerns idiosyncratic DILI and not intrinsic DILI caused of drugs such as paracetamol. The time from initiation of a new drug to the development of liver injury can be hours to months and the manifestation can range from asymptomatic abnormal liver test to acute liver failure³.

The diagnosis must therefore be suspected in people who recently have started on a new drug and no other causes are more evident. DILI is divided into the type of liver cells mainly injured.

The different drugs may cause different patterns of liver injury and the pathogenesis is probably not in common. However, the gut microbiome may impact the susceptibility and outcome to DILI¹¹. Idiosyncratic DILI is managed by discontinuation of the suspected drug, but no specific treatments or prophylaxis is available.

### Treatment Effectors

Intake of fermented cereal in combination with probiotics has surprisingly been shown to have an advantageous clinical effect in the treatment of a fatty liver or fibrotic liver disease. Results indicate that the fermented cereal compositions according to the present invention also exert postbiotic effects in the treatment of liver diseases.

Probiotic microorganisms have a dual role according to the present invention as the microorganisms are used initially for fermenting a cereal, and later as a treatment effecter when administered to an individual, such as a human being, in need thereof.

Additional treatment effectors in the form of complex lipids, carbohydrates, proteins and other biomolecules originate from the oat gruel and the probiotics employed in the methods of the present invention, and these types of additional treatment effectors are also being produced during the fermentation of the oat gruel.

The addition of lecithin, including any group of phospholipids occurring in animal and plant tissues, to the oat gruel or to the fermented product provide a ready-to-use product containing extra, added materials useful for the production of an effective intestinal mucus layer in individuals in need thereof. The amount of added lecithin would expectedly be higher in a ready-to-use product according to the invention based on fermented cereals having a low content of e.g. phospholipids compared to products based on fermented cereals containing higher amounts of phospholipids.

According to one presently preferred hypothesis, administration of fermented cereal compositions containing probiotic microorganisms, and optionally also lecithin, to individuals, including human beings, suffering from, or at risk of contracting, a fatty or fibrotic liver disease for an appropriate period of time is believed to result in one or more of i) a reduced number and/or a reduced metabolic activity of pro-inflammatory microorganisms of the intestine due to the development of environmental conditions unfavourable to such pro-inflammatory microorganisms.

According to another presently preferred hypothesis, administration for an appropriate period of time of fermented cereal compositions containing probiotic microorganisms, and optionally also lecithin, to individuals, including human beings, suffering from, or at risk of contracting, a fatty or fibrotic liver disease, is believed to result in an increased metabolic activity of anti-inflammatory microorganisms in the intestines and/or in a gradual increase in the number of anti-inflammatory microorganisms in the intestines of an individual due to the continued supply of probiotics and fermented cereals leading to a build up of treatment effectors and a development of a new microbial environment in the intestines.

Additionally, the lecithin of the products of the present invention is believed to provide or aid in the provision of a protective mucus layer in the intestines.

Subsequent to the fermentation process, no further increases in the number of colony forming units (cfu) of probiotic microorganisms can be observed in the by now “ready-to-use” product suitable for formulation as a “dosage form” product suitable for daily intake. Accordingly, the fermented cereal does not serve as a prebiotic compound as it is believed to not stimulate specifically the growth of the probiotic microorganisms in the intestine post fermentation.

The fermented cereal composition preferably has a pH below 5.5 when the fermentation has been completed as a pH below 5.5 is believed to mildly stress and prepare the fermented probiotic microorganisms for cultivation in the stomach and in the intestines of individuals suffering from or at risk of contracting a fatty or fibrotic liver disease.

### Probiotic Microorganisms

The probiotic microorganisms according to the present invention are preferably selected from non-pathogenic and anti-inflammatory lactic acid bacteria.

Lactic acid bacteria useful in fermenting cereals according to the present invention are genera, species, sub-species and strains selected from the group consisting of the genera Carnobacterium, Enterococcus, Lactobacillus, Lactococcus, Leuconostoc, Oenococcus and Pediococcus and Streptococcus.

Lactobacillus species include any one or more species selected from the group consisting of Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp delbrueckii, Lactobacillus delbrueckii subsp. lactis, Lactobacillus durianus, Lactobacillus equi, Lactobacillus farciminis, Lactobacillus ferintoshensis, Lactobacillus fermentum, Lactobacillus fornicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme and Lactobacillus zeae.

One preferred Lactobacillus species is Lactobacillus plantarum, and Lactobacillus plantarum 299 and Lactobacillus plantarum 299v are two preferred Lactobacillus species in accordance with the present invention.

Lactic acid bacteria also include Bifidobacterium species and include in particular any one or more of the following species of Bifidobacteria: Bifidobacterium adolescentis, Bifidobacterium aerophilum, Bifidobacterium angulatum, Bifidobacterium animalis, Bifidobacterium asteroides, Bifidobacterium bifidum, Bifidobacterium boum, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum bv Longum, Bifidobacterium longum bv. Infantis, Bifidobacterium longum bv. Suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, Bifidobacterium urinalis.

According to one presently preferred hypothesis it is believed that probiotic microorganisms according to the present invention should be capable of at least transiently colonizing the intestines of an individual.

The term “at least transiently colonizing the intestine of an individual” shall mean that a microbial organism must be at least transiently present in the intestines following one or more intake(s) of a sufficient amount of the fermented cereal composition according to the invention. An analysis for colonization of the intestines can be carried out e.g. by testing an individual for the presence of non-pathogenic and anti-inflammatory lactic acid bacteria according to the present invention in the faeces following intake of the fermented cereal composition.

The fermented cereal compositions according to the present invention are also available as dried preparations, such as, but not limited to, freeze dried or spray dried preparations. Freeze or spray drying will expectedly result in killing some of the probiotic microorganisms subjected to this form of post-fermentation processing, and this is taken into consideration when formulating the dried preparations.

While it may be possible in one embodiment to add freeze-dried or spray-dried probiotics to both a cereal gruel and/or to the fermented cereal post fermentation, the probiotic microorganisms according to the present invention in other embodiments are preferably not freeze-dried or spray-dried.

However, some probiotic microorganisms according to the present invention may be sufficiently robust to survive a gentle drying step, thereby allowing freeze or spray drying in large numbers. Additionally, some drying protocols may be sufficiently gentle to allow for e.g. spray or freeze dried probiotic microorganisms to be added to the cereal gruel prior to or during fermentation, or added to the fermented cereal composition post fermentation.

The cereal-fermenting probiotic microorganisms of the fermented cereal composition according to the present invention may constitute the only probiotic microorganisms present in the fermented cereal compositions according to the present invention.

However, probiotic microorganisms present in the fermented cereal compositions according to the present invention can also be probiotic microorganisms that are added to the fermented cereal composition.

### Fermentable Cereal Gruels and Fermentation Thereof by Probiotic Microorganisms

The fermentable cereal may be any suitable cereal which can be fermented by probiotic microorganisms according to the present invention and subsequently used as a treatment effecter present in a fermented cereal composition according to the present invention.

The microorganisms in a fermented cereal composition according to the present invention may be any cereal-fermenting probiotic microorganism according to the present invention.

Additionally, probiotic microorganisms according to the present invention that are present in the fermented cereal composition according to the present invention can be added to the fermented cereal composition post fermentation. In the latter case, the cereal-fermenting microorganisms may be alive, attenuated, inactivated or dead when forming part of the fermented cereal composition according to the present invention.

Cereal gruel for use in the preparation of a fermented cereal composition according to the present invention can be prepared essentially as disclosed e.g. in European Patent no. 0 415 941.

The cereal gruel, preferably oat gruel, can for example be prepared by suspending at least about 5 grams of cereal in 100 ml of liquid fermentation medium, such as by suspending at least about 6 grams of cereal in 100 ml of liquid fermentation medium, for example by suspending at least about 7 grams of cereal in 100 ml of liquid fermentation medium, such as by suspending at least about 8 grams of cereal in 100 ml of liquid fermentation medium, for example by suspending at least about 9 grams of cereal in 100 ml of liquid fermentation medium, such as by suspending at least about 10 grams of cereal in 100 ml of liquid fermentation medium, for example by suspending at least about 11 grams of cereal in 100 ml of liquid fermentation medium, such as by suspending at least about 12 grams of cereal in 100 ml of liquid fermentation medium, for example by suspending at least about 13 grams of cereal in 100 ml of liquid fermentation medium, such as by suspending at least about 14 grams of cereal in 100 ml of liquid fermentation medium, for example by suspending at least about 15 grams of cereal in 100 ml of liquid fermentation medium, such as by suspending at least about 16 grams of cereal in 100 ml of liquid fermentation medium, for example by suspending at least about 17 grams of cereal in 100 ml of liquid fermentation medium, such as by suspending at least about 18 grams of cereal in 100 ml of liquid fermentation medium, for example by suspending at least about 19 grams of cereal in 100 ml of liquid fermentation medium, such as by suspending at least about 20 grams of cereal in 100 ml of liquid fermentation medium, for example by suspending at least about 21 grams of cereal in 100 ml of liquid fermentation medium, such as by suspending at least about 22 grams of cereal in 100 ml of liquid fermentation medium, for example by suspending at least about 23 grams of cereal in 100 ml of liquid fermentation medium, such as by suspending at least about 24 grams of cereal in 100 ml of liquid fermentation medium, for example by suspending at least about 25 grams of cereal in 100 ml of liquid fermentation medium, such as by suspending at the most about 30 grams of cereal in 100 ml of liquid fermentation medium.

The liquid fermentation medium is preferably subjected to a heat treatment, such as a pasteurisation, prior to addition of cereal-fermenting microorganisms to the cereal gruel.

The fermented cereal composition according to the present invention is produced by adding cereal-fermenting microorganisms to the cereal gruel and fermenting the cereal gruel until the fermented cereal composition has a pH of preferably less than 5.5, such as preferably less than 5.4, for example preferably less than 5.3, such as preferably less than 5.2, for example preferably less than 5.1, such as preferably less than 5.0, for example preferably less than 4.9, such as preferably less than 4.8, for example preferably less than 4.7, such as preferably less than 4.6, for example preferably less than 4.5, such as preferably less than 4.4, for example preferably less than 4.3, such as preferably less than 4.2, for example preferably less than 4.1, such as preferably less than 4.0, for example preferably less than 3.9, such as preferably less than 3.8, for example preferably less than 3.7, such as preferably less than 3.6.

The fermentation of the cereal gruel takes place at an optimal temperature for at least about 12 hours, such as at least about 15 hours, for example at least about 18 hours, such as at least about 21 hours, such as at least about 24 hours, for example at least about 27 hours, such as at least about 30 hours, for example at least about 33 hours, or until such time as the number of colony forming units (cfu) of the microbial culture ceases to increase.

Prior to the fermentation, the cereal gruel can be supplemented with malted flour, such as malted barley flour, and/or enzyme(s), such as e.g. cellulases, hemi-cellulases and similar cellulose degrading enzymes, including amylases, or/and the cereal gruel may be supplemented with a desirable and sufficient amount of nutrients including energy and carbon sources capable of supporting the growth of the cereal-fermenting microorganisms, including in one embodiment probiotic cereal-fermenting microorganisms.

Prior to, during or following the fermentation of the cereal gruel, the cereal gruel or the fermented composition can also be treated physically/mechanically or otherwise e.g. to reduce the viscosity, to improve the availability of nutrient components, to break down certain cereal molecules, to change the composition of macromolecules and building block molecules, or to improve the shelf life of the fermented cereal composition.

Additionally, phospholipids, proteins, amino acids and/or fibres and any additional component believed to be able to improve the mucus barrier in individuals suffering from a fatty or fibrotic liver disease may be added to the cereal gruel prior to or during fermentation thereof. Said components may also be added directly to the fermented cereal composition post fermentation.

One or more vitamins as well as mineral sources, including B-vitamins and zinc, can also be added.

In preferred embodiments, the cereal gruel is oat gruel.

### Fermented Cereal Compositions

In some embodiments of the present invention, the efficient amount of probiotic microorganisms present in the fermented cereal compositions is preferably at least about 1.0×10⁸ colony forming units (cfu) per millilitre of fermented cereal composition, such as at least about 1.2×10⁸ colony forming units (cfu) per millilitre of fermented cereal composition, for example at least about 1.4×10⁸ colony forming units (cfu) per millilitre of fermented cereal composition, such as at least about 1.6×10⁸ colony forming units (cfu) per millilitre of fermented cereal composition, for example at least about 1.8×10⁸ colony forming units (cfu) per millilitre of fermented cereal composition, such as at least about 2.0×10⁸ colony forming units (cfu) per millilitre of fermented cereal composition, for example at least about 3.0×10⁸ colony forming units (cfu) per millilitre of fermented cereal composition, such as at least about 4.0×10⁸ colony forming units (cfu) per millilitre of fermented cereal composition, for example at least about 5.0×10⁸ colony forming units (cfu) per millilitre of fermented cereal composition, such as at least about 6.0×10⁸ colony forming units (cfu) per millilitre of fermented cereal composition, for example at least about 7.0×10⁸ colony forming units (cfu) per millilitre of fermented cereal composition, such as at least about 8.0×10⁸ colony forming units (cfu) per millilitre of fermented cereal composition, for example at least about 9.0×10⁸ colony forming units (cfu) per millilitre of fermented cereal composition, such as at least about 1.0×10⁹ colony forming units (cfu) per millilitre of fermented cereal composition, for example at least 2.0×10⁹ colony forming units (cfu) per millilitre of fermented cereal composition, such as at least about 3.0×10⁹ colony forming units (cfu) per millilitre of fermented cereal composition, for example at least 4.0×10⁹ colony forming units (cfu) per millilitre of fermented cereal composition, such as at least about 5.0×10⁹ colony forming units (cfu) per millilitre of fermented cereal composition, for example at least 6.0×10⁹ colony forming units (cfu) per millilitre of fermented cereal composition, such as at least about 7.0×10⁹ colony forming units (cfu) per millilitre of fermented cereal composition, for example at least 8.0×10⁹ colony forming units (cfu) per millilitre of fermented cereal composition, such as at least about 9.0×10⁹ colony forming units (cfu) per millilitre of fermented cereal composition, for example at least 1.0×10¹⁰ colony forming units (cfu) per millilitre of fermented cereal composition, and preferably less than 1.0×10¹¹ colony forming units (cfu) per millilitre of fermented cereal composition. The number of colony forming units (cfu) will vary depending on fermentation conditions and depending on the probiotic microorganisms used for the fermentation of the cereal gruel.

Independently of, or in combination with the above-cited number of colony forming units, and when the fermented cereal composition is provided as a liquid composition, preferably as a liquid composition of oat meal fermented by probiotic microorganisms according to the present invention, the fermented cereal composition preferably comprises at least about 5 grams of cereal (dry weight) per 100 ml of liquid fermented cereal composition, such as at least about 6 grams of cereal (dry weight) per 100 ml of liquid fermented cereal composition, for example at least about 7 grams of cereal (dry weight) per 100 ml of liquid fermented cereal composition, such as at least about 8 grams of cereal (dry weight) per 100 ml of liquid fermented cereal composition, for example at least about 9 grams of cereal (dry weight) per 100 ml of liquid fermented cereal composition, such as at least about 10 grams of cereal (dry weight) per 100 ml of liquid fermented cereal composition, for example at least about 11 grams of cereal (dry weight) per 100 ml of liquid fermented cereal composition, such as at least about 12 grams of cereal (dry weight) per 100 ml of liquid fermented cereal composition, for example at least about 13 grams of cereal (dry weight) per 100 ml of liquid fermented cereal composition, such as at least about 14 grams of cereal (dry weight) per 100 ml of liquid fermented cereal composition, for example at least about 15 grams of cereal (dry weight) per 100 ml of liquid fermented cereal composition, such as at least about 16 grams of cereal (dry weight) per 100 ml of liquid fermented cereal composition, for example at least about 17 grams of cereal (dry weight) per 100 ml of liquid fermented cereal composition, such as at least about 18 grams of cereal (dry weight) per 100 ml of liquid fermented cereal composition, for example at least about 19 grams of cereal (dry weight) per 100 ml of liquid fermented cereal composition, such as at least about 20 grams of cereal (dry weight) per 100 ml of liquid fermented cereal composition, for example at least about 21 grams of cereal (dry weight) per 100 ml of liquid fermented cereal composition, such as at least about 22 grams of cereal (dry weight) per 100 ml of liquid fermented cereal composition, for example at least about 23 grams of cereal (dry weight) per 100 ml of liquid fermented cereal composition, such as at least about 24 grams of cereal (dry weight) per 100 ml of liquid fermented cereal composition, for example at least about 25 grams of cereal (dry weight) per 100 ml of liquid fermented cereal composition, such as at the most about 30 grams of cereal (dry weight) per 100 ml of liquid fermented cereal composition.

In one embodiment of the present invention, there is provided a fermented cereal composition comprising
i) at least about 12.5 grams (dry weight) of fermented cereal; and
ii) at least about 1.0×10¹⁰ colony forming units (cfu) of Lactobacillus plantarum cells;
   for use in a method for the treatment of an individual suffering from a fatty liver or fibrotic liver disease.

The Lactobacillus plantarum cells are preferably selected from Lactobacillus plantarum 299 and Lactobacillus plantarum 299v, and said Lactobacillus plantarum cells are preferably used for fermenting a cereal gruel to provide a fermented cereal composition according to the present invention.

In one embodiment of the above-cited aspect of the present invention, the fermented cereal composition is in the form of a ready-to-use solid or liquid formulation, including a formulation contained in a disposable packaging or container.

The treatment comprises a continued administration over a predetermined period of time, including a daily or weekly administration of the fermented cereal composition to the individual to be treated. The weekly administration can include administration of the composition several times during a week, including every second day of a week.

The treatment period is determined at least in part by the individual to be treated. Treatment periods typically range from a few weeks to longer periods of many weeks, such as e.g. 14 weeks, 26 weeks, or longer, including 52 weeks, or more than one year. In one embodiment, the treatment may be repeated after a period with no treatment.

Periods with treatment and no treatment can be adjusted depending on the state of health and/or symptoms or disease indicators of the individual suffering from a fatty or fibrotic liver disease, or at risk of contracting a fatty or fibrotic liver disease.

When there is provided a fermented cereal composition comprising
i) at least about 12.5 grams (dry weight) of fermented cereal; and
ii) at least about 1.0×10¹⁰ colony forming units (cfu) of Lactobacillus plantarum cells;
   it will be understood that the relative amounts and numbers of fermented cereal (dry weight) and colony forming units (cfu) of Lactobacillus plantarum cells, respectively will generally correlate with each other.

Certain deviations among relative amounts and numbers of fermented cereal (dry weight) and colony forming units (cfu) of Lactobacillus plantarum cells, respectively, are allowed, and it will be understood that while the number of colony forming units (cfu) of Lactobacillus plantarum cells will generally be about 1.0×10¹⁰ colony forming units (cfu) of Lactobacillus plantarum cells per about 12.5 gram (dry weight) of fermented cereal, certain deviations from the ratio of about 0.08×10¹⁰ colony forming units (cfu) of Lactobacillus plantarum cells per gram (dry weight) of fermented cereal also fall within the scope of the present invention.

In embodiments of the above-cited aspects of the present invention, fermented cereal compositions are provided, wherein the ratio of colony forming units (cfu) of Lactobacillus plantarum cells to fermented cereal (dry weight) is in the range of from about 0.02×10¹⁰ colony forming units (cfu) of Lactobacillus plantarum cells per gram (dry weight) of fermented cereal to about 0.32×10¹⁰ colony forming units (cfu) of Lactobacillus plantarum cells per gram (dry weight) of fermented cereal; such as from about 0.04×10¹⁰ colony forming units (cfu) of Lactobacillus plantarum cells per gram (dry weight) of fermented cereal to about 0.16×10¹⁰ colony forming units (cfu) of Lactobacillus plantarum cells per gram (dry weight) of fermented cereal.

The above-cited ratios can be higher or lower, respectively, such as from about 0.02×10¹⁰ colony forming units (cfu) of Lactobacillus plantarum cells per gram (dry weight) of fermented cereal to about 0.04×10¹⁰ or 0.08×10¹⁰ colony forming units (cfu) of Lactobacillus plantarum cells per gram (dry weight) of fermented cereal; such as from about 0.08×10¹⁰ colony forming units (cfu) of Lactobacillus plantarum cells per gram (dry weight) of fermented cereal to about 0.16×10¹⁰ or 0.32×10¹⁰ colony forming units (cfu) of Lactobacillus plantarum cells per gram (dry weight) of fermented cereal.

In one embodiment, a ratio of from about 0.02×10¹⁰ to about 0.32×10¹⁰ colony forming units (cfu) of Lactobacillus plantarum cells per gram (dry weight) of fermented cereal also fall within the scope of the present invention.

In one embodiment, a fermented cereal composition comprising a combination of more than at least about 12.5 grams (dry weight) of fermented cereal; and more than at least about 1.0×10¹⁰ colony forming units (cfu) of Lactobacillus plantarum cells is provided in accordance with the methods of the present invention. The cereal composition is preferably provided as a dosage form suitable for a daily intake, including a daily dosage form packaged in a suitable container.

In equally preferred embodiments of the present invention there are provided fermented cereal compositions comprising:
A combination of more than at least about 18.8 grams (dry weight) of fermented cereal, and more than at least about 1.5×10¹⁰ colony forming units (cfu) of Lactobacillus plantarum cells;
a combination of more than at least about 25.0 grams (dry weight) of fermented cereal, and more than at least about 2.0×10¹⁰ colony forming units (cfu) of Lactobacillus plantarum cells;
a combination of more than at least about 31.3 grams (dry weight) of fermented cereal, and more than at least about 2.5×10¹⁰ colony forming units (cfu) of Lactobacillus plantarum cells;
a combination of more than at least about 37.5 grams (dry weight) of fermented cereal, and more than at least about 3.0×10¹⁰ colony forming units (cfu) of Lactobacillus plantarum cells;
a combination of more than at least about 43.8 grams (dry weight) of fermented cereal, and more than at least about 3.5×10¹⁰ colony forming units (cfu) of Lactobacillus plantarum cells;
a combination of more than at least about 50.0 grams (dry weight) of fermented cereal, and more than at least about 4.0×10¹⁰ colony forming units (cfu) of Lactobacillus plantarum cells;
a combination of more than at least or preferably less than about 100 grams (dry weight) of fermented cereal, and more than at least or preferably less than about 8.0×10¹⁰ colony forming units (cfu) of Lactobacillus plantarum cells;
a combination of more than at least or preferably less than about 250 grams (dry weight) of fermented cereal, and more than at least or preferably less than about 2.0×10¹¹ colony forming units (cfu) of Lactobacillus plantarum cells; as well as
a combination of preferably less than about 500 grams (dry weight) of fermented cereal, and preferably less than about 4.0×10¹¹ colony forming units (cfu) of Lactobacillus plantarum cells.

In another embodiment of the present invention, there is provided a fermented cereal composition comprising
i) at least about 4.0 grams (dry weight) of fermented cereal; and
ii) at least about 1.0×10¹⁰ colony forming units (cfu) of Lactobacillus plantarum cells;
   for use in a method for the treatment of an individual suffering from a fatty liver or fibrotic liver disease.

The Lactobacillus plantarum cells are preferably selected from Lactobacillus plantarum 299 and Lactobacillus plantarum 299v, and said Lactobacillus plantarum cells are preferably used for fermenting a cereal gruel to provide a fermented cereal composition according to the present invention.

In one embodiment of the above-cited aspect of the present invention, the fermented cereal composition is in the form of a ready-to-use solid or liquid formulation, including a formulation contained in a disposable packaging or container.

The treatment comprises a continued administration over a predetermined period of time, including a daily or weekly administration of the fermented cereal composition to the individual to be treated. The weekly administration can include administration of the composition several times during a week, including every second day of a week.

The treatment period is determined at least in part by the individual to be treated. Treatment periods typically range from a few weeks to longer periods of many weeks, such as e.g. 14 weeks, 26 weeks, or longer, including 52 weeks, or more than one year. In one embodiment, the treatment may be repeated after a period with no treatment.

Periods with treatment and no treatment can be adjusted depending on the state of health and/or symptoms or disease indicators of the individual suffering from a fatty or fibrotic liver disease, or at risk of contracting a fatty or fibrotic liver disease.

When there is provided a fermented cereal composition comprising
i) at least about 4.0 grams (dry weight) of fermented cereal; and
ii) at least about 1.0×10¹⁰ colony forming units (cfu) of Lactobacillus plantarum cells;
   it will be understood that the relative amounts and numbers of fermented cereal (dry weight) and colony forming units (cfu) of Lactobacillus plantarum cells, respectively will generally correlate with each other.

Certain deviations among relative amounts and numbers of fermented cereal (dry weight) and colony forming units (cfu) of Lactobacillus plantarum cells, respectively, are allowed, and it will be understood that while the number of colony forming units (cfu) of Lactobacillus plantarum cells will generally be about 1.0×10¹⁰ colony forming units (cfu) of Lactobacillus plantarum cells per about 4.0 gram (dry weight) of fermented cereal, certain deviations from the ratio of about 0.25×10¹⁰ colony forming units (cfu) of Lactobacillus plantarum cells per gram (dry weight) of fermented cereal also fall within the scope of the present invention.

In exemplary embodiments of the above-cited aspects of the present invention, fermented cereal compositions are provided, wherein the ratio of colony forming units (cfu) of Lactobacillus plantarum cells to fermented cereal (dry weight) is in the range of from about 0.06×10¹⁰ colony forming units (cfu) of Lactobacillus plantarum cells per gram (dry weight) of fermented cereal to about 1×10¹⁰ colony forming units (cfu) of Lactobacillus plantarum cells per gram (dry weight) of fermented cereal; such as from about 0.13×10¹⁰ colony forming units (cfu) of Lactobacillus plantarum cells per gram (dry weight) of fermented cereal to about 0.5×10¹⁰ colony forming units (cfu) of Lactobacillus plantarum cells per gram (dry weight) of fermented cereal.

The above-cited ratios can be higher or lower, respectively, such as from about 0.06×10¹⁰ colony forming units (cfu) of Lactobacillus plantarum cells per gram (dry weight) of fermented cereal to about 0.13×10¹⁰ or 0.25×10¹⁰ colony forming units (cfu) of Lactobacillus plantarum cells per gram (dry weight) of fermented cereal; such as from about 0.25×10¹⁰ colony forming units (cfu) of Lactobacillus plantarum cells per gram (dry weight) of fermented cereal to about 0.5×10¹⁰ or 1.0×10¹⁰ colony forming units (cfu) of Lactobacillus plantarum cells per gram (dry weight) of fermented cereal.

In one embodiment, a ratio of from about 0.06×10¹⁰ to about 1.0×10¹⁰ colony forming units (cfu) of Lactobacillus plantarum cells per gram (dry weight) of fermented cereal also fall within the scope of the present invention.

Accordingly, in accordance with this embodiment of the present invention there is provided a fermented cereal composition comprising a combination of more than at least about 4.0 grams (dry weight) of fermented cereal; and more than at least about 1.0×10¹⁰ colony forming units (cfu) of Lactobacillus plantarum cells. The cereal composition is preferably provided as a dosage form suitable for a daily intake, including a daily dosage form packaged in a suitable container.

In equally preferred embodiments of the present invention there are provided fermented cereal compositions comprising:
A combination of more than at least about 6.0 grams (dry weight) of fermented cereal, and more than at least about 1.5×10¹⁰ colony forming units (cfu) of Lactobacillus plantarum cells;
a combination of more than at least about 8.0 grams (dry weight) of fermented cereal, and more than at least about 2.0×10¹⁰ colony forming units (cfu) of Lactobacillus plantarum cells;
a combination of more than at least about 10.0 grams (dry weight) of fermented cereal, and more than at least about 2.5×10¹⁰ colony forming units (cfu) of Lactobacillus plantarum cells;
a combination of more than at least about 12.0 grams (dry weight) of fermented cereal, and more than at least about 3.0×10¹⁰ colony forming units (cfu) of Lactobacillus plantarum cells;
a combination of more than at least about 14.0 grams (dry weight) of fermented cereal, and more than at least about 3.5×10¹⁰ colony forming units (cfu) of Lactobacillus plantarum cells;
a combination of more than at least about 16.0 grams (dry weight) of fermented cereal, and more than at least about 4.0×10¹⁰ colony forming units (cfu) of Lactobacillus plantarum cells;
a combination of more than at least about 32.0 grams (dry weight) of fermented cereal, and more than at least about 8.0×10¹⁰ colony forming units (cfu) of Lactobacillus plantarum cells;
a combination of more than at least about 48.0 grams (dry weight) of fermented cereal, and more than at least about 1.2×10¹¹ colony forming units (cfu) of Lactobacillus plantarum cells;
a combination of more than at least or preferably less than about 64.0 grams (dry weight) of fermented cereal, and more than at least or preferably less than about 1.6×10¹¹ colony forming units (cfu) of Lactobacillus plantarum cells;
a combination of more than at least or preferably less than about 80.0 grams (dry weight) of fermented cereal, and more than at least or preferably less than about 2.0×10¹¹ colony forming units (cfu) of Lactobacillus plantarum cells; as well as
a combination of preferably less than about 160 grams (dry weight) of fermented cereal, and preferably less than about 4.0×10¹¹ colony forming units (cfu) of Lactobacillus plantarum cells.

Methods for treatment of an individual suffering from, or at risk of contracting, a fatty or fibrotic liver disease, wherein said methods comprise administration of any of the above-cited fermented cereal compositions, are also provided in accordance with the present invention.

The present invention also provides dosage forms suitable for a daily intake, including daily dosage forms, of the fermented cereal compositions according to the present invention.

Accordingly, in a further embodiment of the present invention there is provided a dosage form comprising a fermented cereal composition comprising
i) at least about 12.5 grams (dry weight) of fermented cereal; and
ii) at least about 1.0×10¹⁰ colony forming units (cfu) of Lactobacillus plantarum cells;
   for use in a method for the treatment of an individual suffering from a fatty or fibrotic liver disease.

The Lactobacillus plantarum cells are preferably selected from Lactobacillus plantarum 299 and Lactobacillus plantarum 299v, and said Lactobacillus plantarum cells are preferably used for fermenting a cereal gruel to provide a fermented cereal composition according to the present invention.

In one embodiment of the above-cited aspect of the present invention, the fermented cereal composition is in the form of a ready-to-use solid or liquid formulation, including a formulation contained in a disposable packaging or container.

The treatment comprises a continued administration over a predetermined period of time, including a daily or weekly administration of the fermented cereal composition to the individual to be treated. The weekly administration can include administration of the composition several times during a week, including every second day of a week.

The treatment period is determined at least in part by the individual to be treated. Treatment periods typically range from a few weeks to longer periods of many weeks, such as e.g. 14 weeks, 26 weeks, or longer, including 52 weeks, or more than one year. In one embodiment, the treatment may be repeated after a period with no treatment. Periods with treatment and no treatment can be adjusted depending on the state of health and/or symptoms, if any, or disease indicators of the individual suffering from a fatty or fibrotic liver disease, or at risk of contracting a fatty or fibrotic liver disease.

When there is provided a dosage form comprising a fermented cereal composition comprising
iii) at least about 12.5 grams (dry weight) of fermented cereal; and
iv) at least about 1.0×10¹⁰ colony forming units (cfu) of Lactobacillus plantarum cells;
   it will be understood that the relative amounts and numbers of fermented cereal (dry weight) and colony forming units (cfu) of Lactobacillus plantarum cells, respectively will generally correlate with each other.

Certain deviations among relative amounts and numbers of fermented cereal (dry weight) and colony forming units (cfu) of Lactobacillus plantarum cells, respectively, are allowed, as cited herein above. In one embodiment, the dosage forms comprise fermented cereal compositions having a ratio of from about 0.04×10¹⁰ to about 0.32×10¹⁰ colony forming units (cfu) of Lactobacillus plantarum cells per gram (dry weight) of fermented cereal.

Dosage forms suitable for a daily intake comprising fermented cereal compositions comprising a combination of more than at least about 12.5 grams (dry weight) of fermented cereal; and more than at least about 1.0×10¹⁰ colony forming units (cfu) of Lactobacillus plantarum cells are also provided in accordance with the present invention.

The dosage form is preferably for use in a method for the treatment of an individual suffering from a fatty or fibrotic liver disease; wherein the treatment comprises administration daily or several times weekly, such as from 2 to 4 times weekly, or at least 5 times weekly, of the fermented cereal composition to the individual to be treated.

In equally preferred embodiments of the present invention there are provided a dosage form comprising fermented cereal compositions comprising:
A combination of more than at least about 18.8 grams (dry weight) of fermented cereal, and more than at least about 1.5×10¹⁰ colony forming units (cfu) of Lactobacillus plantarum cells;
a combination of more than at least about 25.0 grams (dry weight) of fermented cereal, and more than at least about 2.0×10¹⁰ colony forming units (cfu) of Lactobacillus plantarum cells;
a combination of more than at least about 31.3 grams (dry weight) of fermented cereal, and more than at least about 2.5×10¹⁰ colony forming units (cfu) of Lactobacillus plantarum cells;
a combination of more than at least about 37.5 grams (dry weight) of fermented cereal, and more than at least about 3.0×10¹⁰ colony forming units (cfu) of Lactobacillus plantarum cells;
a combination of more than at least about 43.8 grams (dry weight) of fermented cereal, and more than at least about 3.5×10¹⁰ colony forming units (cfu) of Lactobacillus plantarum cells;
a combination of more than at least about 50.0 grams (dry weight) of fermented cereal, and more than at least about 4.0×10¹⁰ colony forming units (cfu) of Lactobacillus plantarum cells;
a combination of more than at least or preferably less than about 100 grams (dry weight) of fermented cereal, and more than at least or preferably less than about 8.0×10¹⁰ colony forming units (cfu) of Lactobacillus plantarum cells;
a combination of more than at least or preferably less than about 250.0 grams (dry weight) of fermented cereal, and more than at least or preferably less than about 2.0×10¹¹ colony forming units (cfu) of Lactobacillus plantarum cells; as well as
a combination of preferably less than about 500 grams (dry weight) of fermented cereal, and preferably less than about 4.0×10¹¹ colony forming units (cfu) of Lactobacillus plantarum cells.

In a still further embodiment of the present invention there is provided a dosage form comprising a fermented cereal composition comprising
i) at least about 4.0 grams (dry weight) of fermented cereal; and
ii) at least about 1.0×10¹⁰ colony forming units (cfu) of Lactobacillus plantarum cells;
   for use in a method for the treatment of an individual suffering from a fatty or fibrotic liver disease.

The Lactobacillus plantarum cells are preferably selected from Lactobacillus plantarum 299 and Lactobacillus plantarum 299v, and said Lactobacillus plantarum cells are preferably used for fermenting a cereal gruel to provide a fermented cereal composition according to the present invention.

In one embodiment of the above-cited aspect of the present invention, the fermented cereal composition is in the form of a ready-to-use solid or liquid formulation, including a formulation contained in a disposable packaging or container.

The treatment comprises a continued administration over a predetermined period of time, including a daily or weekly administration of the fermented cereal composition to the individual to be treated. The weekly administration can include administration of the composition several times during a week, including every second day of a week.

The treatment period is determined at least in part by the individual to be treated. Treatment periods typically range from a few weeks to longer periods of many weeks, such as e.g. 14 weeks, 26 weeks, or longer, including 52 weeks, or more than one year. In one embodiment, the treatment may be repeated after a period with no treatment.

Periods with treatment and no treatment can be adjusted depending on the state of health and/or symptoms or disease indicators of the individual suffering from a fatty or fibrotic liver disease, or at risk of contracting a fatty or fibrotic liver disease.

When there is provided a dosage form suitable for a daily intake comprising a fermented cereal composition comprising
i) at least about 4.0 grams (dry weight) of fermented cereal; and
ii) at least about 1.0×10¹⁰ colony forming units (cfu) of Lactobacillus plantarum cells;
   it will be understood that the relative amounts and numbers of fermented cereal (dry weight) and colony forming units (cfu) of Lactobacillus plantarum cells, respectively will generally correlate with each other.

Certain deviations among relative amounts and numbers of fermented cereal (dry weight) and colony forming units (cfu) of Lactobacillus plantarum cells, respectively, are allowed as disclosed herein above. In one embodiment, dosage form compositions having a ratio of from about 0.06×10¹⁰ to about 1.0×10¹⁰ colony forming units (cfu) of Lactobacillus plantarum cells per gram (dry weight) of fermented cereal also fall within the scope of the present invention.

In accordance with this embodiment of the present invention there is provided a dosage form comprising fermented cereal compositions suitable for daily intake comprising a combination of more than at least about 4.0 grams (dry weight) of fermented cereal; and more than at least about 1.0×10¹⁰ colony forming units (cfu) of Lactobacillus plantarum cells.

The dosage form is preferably for use in a method for the treatment of an individual suffering from a fatty or fibrotic liver disease; wherein the treatment comprises administration daily or several times weekly, such as from 2 to 4 times weekly, for example at least 5 times weekly, of the fermented cereal composition to the individual to be treated.

In equally preferred embodiments of the present invention there are provided dosage forms suitable for daily intake comprising fermented cereal compositions comprising:
A combination of more than at least about 6.0 grams (dry weight) of fermented cereal, and more than at least about 1.5×10¹⁰ colony forming units (cfu) of Lactobacillus plantarum cells;
a combination of more than at least about 8.0 grams (dry weight) of fermented cereal, and more than at least about 2.0×10¹⁰ colony forming units (cfu) of Lactobacillus plantarum cells;
a combination of more than at least about 10.0 grams (dry weight) of fermented cereal, and more than at least about 2.5×10¹⁰ colony forming units (cfu) of Lactobacillus plantarum cells;
a combination of more than at least about 12.0 grams (dry weight) of fermented cereal, and more than at least about 3.0×10¹⁰ colony forming units (cfu) of Lactobacillus plantarum cells;
a combination of more than at least about 14.0 grams (dry weight) of fermented cereal, and more than at least about 3.5×10¹⁰ colony forming units (cfu) of Lactobacillus plantarum cells;
a combination of more than at least about 16.0 grams (dry weight) of fermented cereal, and more than at least about 4.0×10¹⁰ colony forming units (cfu) of Lactobacillus plantarum cells;
a combination of more than at least about 32.0 grams (dry weight) of fermented cereal, and more than at least about 8.0×10¹⁰ colony forming units (cfu) of Lactobacillus plantarum cells;
a combination of more than at least about 48.0 grams (dry weight) of fermented cereal, and more than at least about 1.2×10¹¹ colony forming units (cfu) of Lactobacillus plantarum cells;
a combination of more than at least or preferably less than about 64.0 grams (dry weight) of fermented cereal, and more than at least or preferably less than about 1.6×10¹¹ colony forming units (cfu) of Lactobacillus plantarum cells;
a combination of more than at least or preferably less than about 80.0 grams (dry weight) of fermented cereal, and more than at least or preferably less than about 2.0×10¹¹ colony forming units (cfu) of Lactobacillus plantarum cells; as well as
a combination of preferably less than about 160 grams (dry weight) of fermented cereal, and preferably less than about 4.0×10¹¹ colony forming units (cfu) of Lactobacillus plantarum cells.

In one embodiment of the present invention there is provided a dosage form of a fermented cereal composition comprising at least 18 grams (dry weight) of fermented cereal; and at least 1×10¹¹ colony forming units (cfu) of Lactobacillus plantarum cells preferably selected from Lactobacillus plantarum 299 and Lactobacillus plantarum 299v; for use in a method for the treatment of an individual suffering from a fatty or fibrotic liver disease; wherein the treatment comprises administration daily or several times weekly, such as from 2 to 4 times weekly, of the fermented cereal composition to the individual to be treated.

The dosage forms suitable for daily intake and fermented cereal compositions in other embodiments comprise at least 36 gram (g) (dry weight) of fermented cereal and at least 2×10¹¹ colony forming units (cfu) of said Lactobacillus plantarum cells, such as at least 90 grams (dry weight) of fermented cereal and at least 5×10¹¹ colony forming units (cfu) of said Lactobacillus plantarum cells.

The dosage forms and fermented cereal compositions preferably comprises fermented cereal in the form of fermented oat meal, and the oat meal is preferably present in the composition in amounts of at least 0.05 gram (dry weight) per milliliter of the composition, such as at least 0.1 gram (dry weight) per milliliter (ml) of the composition, for example at least 0.18 gram (dry weight) per milliliter (ml) of the composition.

In some embodiments, the dosage forms and fermented cereal compositions according to the present invention preferably comprise more than about 1×10⁸ colony forming units (cfu) of Lactobacillus plantarum cells per milliliter (ml) of the fermented cereal composition, such as more than about 1×10⁹ colony forming units (cfu) of Lactobacillus plantarum cells are present per milliliter (ml) of the fermented cereal composition, for example more than, or less than, about 1×10¹⁰ colony forming units (cfu) of Lactobacillus plantarum cells are present per milliliter (ml) of the fermented cereal composition.

These dosage forms are also preferably for use in a method for the treatment of an individual suffering from a fatty or fibrotic liver disease; wherein the treatment comprises administration daily or several times weekly, such as from 2 to 4 times weekly, for example at least 5 times weekly, of the fermented cereal composition to the individual to be treated.

It is preferred that the fermented cereal composition is obtained by fermenting a cereal gruel with said Lactobacillus plantarum cells, but said Lactobacillus plantarum cells can also be added to the fermented cereal post fermentation.

The dosage forms suitable for daily intake and fermented cereal compositions according to the present invention can contain added phospholipids, such as lecithin or phosphatidylcholine, in addition to any phospholipids natively occurring in the cereal gruel, preferably oat gruel or oat meal.

In embodiments of the present invention when phospholipids are present in the fermented cereal compositions, the phospholipids preferably comprise one or more of phosphatidyl-choline (PC), lyso-phosphatidyl-choline (LPC), phosphatidyl-inositol (PI) and/or phosphatidyl-ethanolamine (PE).

Fermented cereal compositions according to the present invention comprising phospholipids are believed according to one presently preferred hypothesis to be preferred when treating individuals suffering from or at risk of contracting a fatty or fibrotic liver disease.

The term “lecithin” as used herein refers both to the chemical definition, which is phosphatidyl-choline, or 1,2-diacyl-glycero-3-phospho-choline, and to a generally used commercial definition, which refers to a natural mixture of neutral and polar lipids. The term “lecithin” includes chemically or commercially defined lecithin in any physical form, including liquid, granulated, and encapsulated forms, as well as combinations of lecithin with any other substance.

Phosphatidyl-choline is present in commercial lecithin in amounts of from about 20% and up to about or more than 90%. Most commercial lecithin products contain about 20% phosphatidyl-choline. Other components in commercial lechithin are lyso-phosphatidyl-choline (LPC), phosphatidyl-inositol (PI) and phosphatidyl-ethanolamine (PE). The fatty acid residues of phosphatidylcholine can be saturated, mono-unsaturated or poly-unsaturated.

In some embodiments, the dosage forms and fermented cereal compositions according to the present invention do not contain milk components, including lactose and milk proteins, as well as other fermentable sugars, such as sucrose, glucose and fructose.

Minerals can also be present in the compositions according to the present invention, and the term “mineral” as used herein comprises pharmaceutical acceptable minerals such as chrome, iron, zinc, copper, calcium, potassium, sodium, manganese and molybdenum. Minerals can be included in the compositions based on a daily administration of e.g. from 20 to 1000 mg, or higher, of each mineral.

The invention can also be combined with a vitamin and/or co-factor supplement or treatment, or formulated in a kit-of-parts further comprising one or more vitamins and/or co-factors in predetermined dosages.

The vitamins in question and their preferred daily dosages can e.g. be Vitamin B: Thiamin (B1) (e.g. from 14 to 30 mg daily dosage), Riboflavin (B2) (e.g. from 11 to 24 mg daily dosage), Vitamin B6 (e.g. from 5 to 11 mg daily dosage), Vitamin B12 (e.g. from 9 to 18 μg daily dosage), Folic acic (e.g. from 180 to 400 μg daily dosage), Niacin (e.g. from 60 to 120 mg daily dosage), Pantothen acid (e.g. from 27 to 54 mg daily dosage), and Biotin (e.g. from 50 to 100 μg daily dosage).

Additionally, Curcumin can be present based on a daily intake of from 3 g to 12 g.

The invention can also be combined with an antibiotic treatment, or formulated in a kit-of-parts further comprising one or more antibiotics in predetermined dosages.

The antibiotics include in particular gut-specific antibiotics, including rifaximin, vancomycin, one or more quinolones (e.g. ciprofloxacin and/or norfloxacin) and metronidazole. Particularly preferred are gut-specific antibiotics such as rifaximin which is poorly absorbed from the gut.

Recommended daily dosages of selected antibiotics are:
Rifaximin: From about 600 mg (traveller's diarrhea), to about 1100 mg (coma due to liver disease.
Vancomycin: From about 500 mg to about 2000 mg.
Ciprofloxacin: From about 500 mg to about 1000 mg.
Metronidazole: From about 1000 mg to about 2000 mg.

A skilled person will know which further additives to add to the compositions according to the present invention.

### Suitable Amounts for Daily Intake and Recommended Daily Dosage Forms

When there is provided a dosage form comprising a fermented cereal composition comprising
i) at least about 4.0 grams (dry weight) of fermented cereal; and
ii) at least about 1.0×10¹⁰ colony forming units (cfu) of Lactobacillus plantarum cells;
   for use in a method for the treatment of an individual suffering from a fatty or fibrotic liver disease, said dosage form is in one embodiment a daily dosage form.

In further embodiments, the present invention is directed to dosage forms and fermented cereal compositions for daily intake containing at least about 10 grams by dry weight of a fermented cereal—for example fermented oat gruel—and at least about 2.5×10¹⁰ colony forming units (cfu) of probiotic microorganisms—for example lactic acid bacteria, such as intestine colonizing Lactobacillus species, including Lactobacillus plantarum.

Suitable amounts for daily intake can also be e.g. at least about 18 g dry weight of fermented cereal, e.g. fermented oat gruel, and 1×10¹¹ cfu of probiotic microorganisms, or at least about 36 g dry weight of fermented cereal, e.g. fermented oat gruel, and 2×10¹¹ cfu of probiotic microorganisms, or at least about 90 g dry weight of fermented cereal, e.g. fermented oat gruel, and about 5×10¹¹ cfu of probiotic microorganisms, such as intestine colonizing Lactobacillus species, including Lactobacillus plantarum.

Lower or higher amounts of fermented cereal compositions, including e.g. fermented oat gruel compositions, and lower or higher numbers of colony forming units of probiotic microorganisms can be used if desired or necessary.

The daily intake can be divided into one or more daily intakes. The first intake of the ready-to-use product is preferably before breakfast, and the last intake is preferably after the last meal and drink of the day.

The last intake may be chosen to allow the fermented cereal composition to be present in the intestines of an individual for as long as possible without being “diluted” by “normal” foods and drinks. It is acceptable to drink water after the last intake. However, intake of the ready-to-use product in connection with each meal during the day may also be an option.

The duration of the treatment is dependent on each individual, including a human being, receiving treatment, and the duration of the treatment will generally be adjusted to the stage, severity and progression of the fatty or fibrotic liver disease to be treated.

Typical treatment periods range from 1 week to 25 weeks, but there is generally no upper limit of the treatment period. A continued treatment for a certain period of time after remission may in some cases provide better long term effects.

The terms “fermented product” and “ready-to-use fermented product” are defined as a dosage form or a fermented cereal composition to be administered to a patient, including a human being, for instance as a drinkable or eatable composition. Drinkable ready-to-use fermented products may be stored and be readily available in suitable containers.

Other administration routes can also be applied, including anal administration or administration of the compositions according to the invention as tube administrable products. Anally administrable ready-to-use products may for instance be in the form of a suppository, and tube administrable ready-to-use products may for instance be in a liquid form that is suitable for tube administration via the oral or rectal route.

The terms “daily treatment”, “daily administration” and “daily intake” are used interchangeably and shall be understood as the total dose of the (ready-to-use) fermented product to be taken by or administered to an individual each day during a treatment period.

If the treatment is administered by the patient him/herself on a daily basis, the recommended dosage is preferably provided in a suitable container containing the correct daily dosage or sub-dosages to be taken on a daily basis. The total daily dose may advantageously be divided into two or more container compartments containing the recommended dosage to be taken or administered during the day, for example with the first and the last meal of the day.

Alternatively, medical staff may measure the correct, recommended dosage for each administration from a larger container comprising the (ready-to-use) fermentable product.

The present invention can be characterized by the following items:
1. A ready-to-use product comprising a composition comprising
   at least about 12.5 gram (g) (dry weight) of fermented cereal;
   and
   at least about 1.0×10¹⁰ colony forming units (cfu) of Lactobacillus plantarum cells selected from Lactobacillus plantarum 299 and Lactobacillus plantarum 299v;
   for use in a method for the treatment of a patient suffering from a fibrotic liver disease;
   wherein the treatment comprises a daily administration of the composition to the patient.
2. The product for use according to item 1, wherein the composition comprises at least about 25 gram (g) (dry weight) of fermented cereal and at least about 2.0×10¹⁰ colony forming units (cfu) of said Lactobacillus plantarum cells.
3. The product for use according to item 1, wherein the composition comprises at least about 50 gram (g) (dry weight) of fermented cereal and at least about 4.0×10¹⁰ colony forming units (cfu) of said Lactobacillus plantarum cells.
4. The product for use according to any of items 1 to 3, wherein the fermented cereal is oatmeal, and wherein the oatmeal is present in the composition in a concentration of at least 0.05 gram (g) (dry weight) per milliliter (ml) of the composition.
5. The product for use according to any of items 1 to 3, wherein the fermented cereal is oatmeal, and wherein the oatmeal is present in the composition in a concentration of at least 0.1 gram (g) (dry weight) per milliliter (ml) of the composition.
6. The product for use according to any of items 1 to 3, wherein the fermented cereal is oatmeal, and wherein the oatmeal is present in the composition in a concentration of at least 0.18 gram (g) (dry weight) per milliliter (ml) of the composition.
7. The product for use according to any of items 1 to 6, wherein at least 1×10⁸ colony forming units (cfu) of said Lactobacillus plantarum cells are present per milliliter (ml) of the composition.
8. The product for use according to any of items 1 to 6, wherein at least 1×10⁹ colony forming units (cfu) of said Lactobacillus plantarum cells are present per milliliter (ml) of the composition.
9. The product for use according to any of items 1 to 3, wherein the fermented cereal is obtained by fermenting a cereal with one or more of said Lactobacillus plantarum cells.
10. The product for use according to any of items 1 to 3, wherein said one or more Lactobacillus plantarum cells are added to the fermented cereal.
11. The product for use according to any of items 1 to 10, wherein the product comprises added lecithin or added phosphatidylcholine.
12. The product for use according to item 11, wherein the treatment involves administration to said patient of at least 0.02 g added lecithin or added phosphatidylcholine on a daily basis.
13. The product for use according to item 11, wherein the treatment involves administration to said patient of at least 0.05 g added lecithin or added phosphatidylcholine on a daily basis.
14. The product for use according to item 11, wherein the treatment involves administration to said patient of at least 0.1 g added lecithin or added phosphatidylcholine on a daily basis.
15. The product for use according to any of items 1 to 14, wherein the cereal is oat (oatmeal).
16. The product for use according to any of the items 1 to 15, wherein said product does not contain any milk or milk products or fermentable sugars in the form of sucrose, lactose, glucose or fructose.
17. A method of treating a fibrotic liver disease in a human being, the method comprising administering to the human being on a daily basis
   a fermented cereal at least about 12.5 gram (g) (dry weight) of fermented cereal;
   and
   at least about 1.0×10¹⁰ colony forming units (cfu) of Lactobacillus plantarum cells selected from Lactobacillus plantarum 299 and Lactobacillus plantarum 299v;
      wherein the fermented cereal is administered for a period of time effective to reduce at least one symptom or disease marker of a fibrotic liver disease.
18. The method of item 17, wherein the cereal is fermented by said Lactobacillus plantarum cells.
19. The method of item 17, wherein said Lactobacillus plantarum cells are added to the fermented cereal.
20. The method of item 17, where the cereal that is fermented is selected from barley, corn, millet, oats or oatmeal, quinoa, rice, rye, sorghum, triticale, wheat, and cassava.
21. The method of item 17, wherein the cereal that is fermented is oats or oatmeal.
22. The method of item 17, wherein the fermented cereal is administered in an amount providing at least 2.0×10¹⁰ cfu of said Lactobacillus plantarum cells on a daily basis.
23. The method of item 17, wherein the fermented cereal is administered in an amount providing at least 4.0×10¹⁰ cfu of said Lactobacillus plantarum cells on a daily basis.
24. The method of item 22, wherein the fermented cereal is administered in an amount providing at least 25 g (dry weight) of fermented cereal on a daily basis.
25. The method of item 23, wherein the fermented cereal is administered in an amount providing at least 50 g (dry weight) of fermented cereal on a daily basis.
26. The method of item 17, wherein the fermented cereal comprises at least 1×10⁸ cfu/ml of said Lactobacillus plantarum cells.
27. The method of item 17, wherein the fermented cereal comprises at least 1×10⁹ cfu/ml of said Lactobacillus plantarum cells.
28. The method of item 17, wherein the fermented cereal comprises at least 0.05 g/ml (dry weight) of fermented cereal.
29. The method of item 17, wherein the fermented cereal comprises at least 0.1 g/ml (dry weight) fermented cereal.
30. The method of item 17, wherein the fermented cereal comprises at least 0.18 g/ml (dry weight) fermented cereal.
31. The method of item 17, wherein the fermented cereal comprises added lecithin or added phosphatidylcholine and is orally administered.
32. The method of item 17, wherein the fermented cereal is administered in an amount providing at least 0.02 g added lecithin or phosphatidylcholine on a daily basis.
33. The method of item 17, wherein the fermented cereal is administered in an amount providing at least 0.05 g added lecithin or phosphatidylcholine on a daily basis.
34. The method of item 17, wherein the fermented cereal is administered in an amount providing at least 0.1 g added lecithin or phosphatidylcholine on a daily basis.
35. The method of item 17, wherein the fermented cereal further comprises one or more food additives, medical additives, taste enhancers, food colors, pH or osmosis regulators, vitamins, herbs, herbal components, minerals, viscosity regulators, lipids, emulsifiers, glutamine or other amino acids, antioxidants, blood pressure regulators, or pain relief substances.
36. The method of item 17, wherein the fermented cereal further comprises an additional active agent, including an antibiotic, suitable for treatment of liver disorders.
37. The method of item 17, wherein the fermented cereal is administered in the form of a drinkable, eatable, anally administrable, or tube administrable product.

### REFERENCES

1. Louvet A, Mathurin P. Alcoholic liver disease: mechanisms of injury and targeted treatment. Nat Rev Gastroenterol Hepatol 2015;12:231-42.
2. Goel A, Gupta M, Aggarwal R. Gut microbiota and liver disease. Journal of gastroenterology and hepatology 2014;29:1139-48.
3. Leise M D, Poterucha J J, Talwalkar J A. Drug-induced liver injury. Mayo Clinic proceedings 2014;89:95-106.
4. Schuppan D, Afdhal N H. Liver cirrhosis. Lancet 2008;371:838-51.
5. European Association for the Study of L. EASL clinical practical guidelines: management of alcoholic liver disease. Journal of hepatology 2012;57:399-420.
6. O'Shea R S, Dasarathy S, Mccullough A J, Practice Guideline Committee of the American Association for the Study of Liver D, Practice Parameters Committee of the American College of G. Alcoholic liver disease. Hepatology 2010;51:307-28.
7. Thiele M, Detlefsen S, Sevelsted Moller L, et al. Transient and 2-dimensional Shear-Wave Elastography provide comparable assessment of Alcoholic Liver Fibrosis and Cirrhosis. Gastroenterology 2015.
8. Thursz M R, Richardson P, Allison M, et al. Prednisolone or pentoxifylline for alcoholic hepatitis. The New England journal of medicine 2015;372:1619-28.
9. Chalasani N, Younossi Z, Lavine J E, et al. The diagnosis and management of non-alcoholic fatty liver disease: practice guideline by the American Gastroenterological Association, American Association for the Study of Liver Diseases, and American College of Gastroenterology. Gastroenterology 2012;142:1592-609.
10. European Association for the Study of the L. EASL Clinical Practice Guidelines: management of cholestatic liver diseases. Journal of hepatology 2009;51:237-67.
11. Fontana R J. Pathogenesis of idiosyncratic drug-induced liver injury and clinical perspectives. Gastroenterology 2014;146:914-28.
12. Ferenci P, Lockwood A, Mullen K, Tarter R, Weissenborn K, Blei A T. Hepatic encephalopathy—definition, nomenclature, diagnosis, and quantification: final report of the working party at the 11th World Congresses of Gastroenterology, Vienna, 1998. Hepatology 2002;35:716-21.

### EXAMPLES

The following examples are intended to illustrate the present invention and should in no way limit the invention as defined in the claims.

The following examples disclose a treatment of alcoholic liver fibrosis, and the examples will be used as a proof of concept of the present treatment concept and the applicability of the concept for treating other liver diseases in which gut imbalance is believed to be associated with the pathogenesis.

### Example 1

### Manufacture of a Ready-to-Use Product Consisting of Fermented Oat Gruel With Probiotic Intestine Colonizing Lactobacillus Plantarum

The manufacture of a ready-to-use product consisting of fermented oat gruel with probiotic intestine colonizing Lactobacillus plantarum is divided into two steps according to this recipe.

This first step covers the preparation of oat gruel. This step may be performed essentially as disclosed in EP 0 415 941 B1.

An alternative method is to mix 18.5% (w/w) oatmeal and 0.9-2.5% (w/w) malted barley flour with water. The mixture is slowly stirred and heated for 10-20 minutes at 37° C. and following 15-30 minutes at 90-100° C. The resulting oat gruel is cooled to a temperature of about 37° C. and is now ready for the second step namely the fermentation process. A starter culture consisting of Lactobacillus plantarum 299 or strain 299v is added to the oat gruel to initiate the fermentation.

The amount of added starter culture with a cfu number of about 10⁹/ml is 0.01, 0.1 or 1.0% (v/v). The fermentation is carried out with mild stirring at 37° C. for 12-24 hrs.

The resulting ready to use product with a cfu number of at least 10⁸/ml, and a pH below 5.5, preferably a pH below 5.0, is then cooled at 4° C. and packed for instance in sterile storage containers of 250 ml, which have a shelf life of at least two months, and preferably a shelf life of at least about five months, when kept at 4° C.

### Example 2

### Manufacture of a Ready-to-Use Product Consisting of Fermented Oat Gruel With Probiotic Microorganisms and Lecithin

A ready to use product consisting of fermented oat gruel with probiotic microorganisms is manufactured as described in Example 1.

Subsequently, lecithin is added. 12 grams of granulated lecithin, such as e.g. “Lecithin Granulat” from Biosym A/S, DK-7430 Ikast, Denmark, are added per liter fermented oat gruel with probiotic microorganisms.

The mixture is stirred for about one minute and kept overnight at 4° C. resulting in a ready-to-use product comprising fermented oat gruel and probiotic microorganisms and lecithin, wherein the product has a cfu number of at least about 10⁸/ml and pH below 5.5, preferably below 5.0. This product is packed for instance in sterile storage containers of 250 ml, which have a shelf life of at least two months, and preferably a shelf life of at least about five months, when kept at 4° C.

Larger or smaller amounts of lecithin, or other physical forms and qualities of lecithin, can be used in this protocol. Numerous commercial lecithins are available and most have different contents of phosphatidylcholine, lysophosphatidylcholine, phosphatidylinositol and phosphatidylethanolamine.

The fatty acids residues of these phospholipids may be saturated, mono-unsaturated or poly-unsaturated. Also, the lecithin to be used in this protocol may have different physical forms such as liquid, granulated, encapsulated or mixed with any other substances such as vegetable oils. Encapsulated lecithin include formulations where lecithin is designed to be released at specific locations in the gastrointestinal tract for instance as defined as “retarded release” by Stremmel et al. 2005 Gut 54:966-971.

### Example 3

### Clinical Studies on Alcoholic Liver Fibrosis

Using a composition manufactured in Example 1 or 2, a series of case studies is being performed with 4-8 patients suffering from alcoholic liver disease. The composition is hereafter called “fermented oat gruel”.

### Inclusion Criteria

1) A significant alcohol intake≥60 gram/day for at least 1 year
2) Abnormal blood test indicating alcoholic liver disease defined by abnormal levels of at least one of the following parameters: Bilirubin, albumin, coagulation factors, international normalized ratio, mean corpuscular volume (MCV), aspartate aminotransferases (AST), alanine aminotransferases (ALT), alkaline phosphatases, immunoglobuline-A (IgA), gammaglutamyltranspeptidases (GGT)
3) The participants may not change their alcohol habits during the study.

### Exclusion Criteria

1) Other causes of liver disease should be excluded
2) Complete alcohol abstinence or a desire of this.
3) Treatment with antibiotics during the last 8 weeks
4) Pregnancy
5) Cancer

### Intervention

Participants will be allocated to group A) having 250 ml fermented oat gruel daily or group B) having 500 ml fermented oat gruel daily. The fermented oat gruel must be taken daily for at least 26 weeks. The participants may not change their alcohol habits during the study.

### Data Collection

Data will expectedly be collected before entering the study, after 13 weeks, end of treatment, and a follow up at 52 weeks. Following data will be collected:

Blood test will be performed by the participant's GP. Possibly a questionnaire of alcohol craving and nutritional status will be evaluated by the Penn Alcohol Craving Scale and Body mass index (BMI).

Endpoint: Improvement of the abnormal blood test indicating reduced liver damage. Possibly reduction of alcohol craving and improved nutritional status

### Example 4

### Randomized Controlled Study

A randomized controlled study will be performed on about 80 participants diagnosed with moderate to severe alcoholic liver fibrosis.

### Inclusion Criteria

1. Age of 18-80 years
2. A significant alcohol intake>30 gram/day
3. Significant alcoholic liver fibrosis verified by liver biopsy using a cut-off for significant alcoholic liver disease (Ishak Score 3-5 or Metavir F2-3) or by an indirect test which have been validated to evaluate the amount of fibrosis ad the severity of the liver disease (e.g. transient elastography and 2D shear wave)
4. Informed consent

### Exclusion Criteria

1) Other causes of liver disease should be excluded including viral hepatitis, autoimmune hepatitis, hemochromatosis, Wilson's disease, alfa-1 antitrypsin deficiency, drug-induced liver disease.
2) Complete alcohol abstinence or a desire of this.
3) Treatment with antibiotics during the last 8 weeks
4) Pregnancy
5) Cancer

### Randomization and Intervention

Randomization and allocation will be done in the ratio 1:1. Group A will receive fermented oat gruel daily while Group B will receive a control product with about the same volume and about the same energy content and distribution as the fermented oat gruel product. The dose of the intervention will be decided after evaluation of the results of the study described in Example 3. The participants may not change their alcohol habits during the study.

Treatment duration will be at least 26 weeks.

### Data Collection

Data will be collected the entry to the study, after 13 weeks and 26 weeks. Additional data collections will be performed if the treatment duration is prolonged.

Data collection includes: 1) Evaluation of the liver stiffness to assess the amount of fibrosis will be done by transient elastography and 2D shear wave 2) Feces samples for microbiome and metabolome analysis 3) Routine blood samples including bilirubin, albumin, coagulation factors, international normalized ratio, mean corpuscular volume (MCV), aspartate aminotransferases (AST), alanine aminotransferases (ALT), alkaline phosphatases, immunoglobuline-A (IgA), gammaglutamyltranspeptidases (GGT) 4) general health examination by a physician including evaluation of alcohol craving, nutritional status and general well-being.

### Primary Endpoint

A significant reduction of the liver stiffness evaluated by an indirect test, which have been validated to evaluate the amount of fibrosis ad the severity of the liver disease (e.g. transient elastography and 2D shear wave)

### Secondary Endpoints

Microbiotic and/or metabolotic changes in the feces samples indicating a change in the gut flora.
Improvement of routine blood samples
Reduction of alcohol craving
Improvement of nutritional status
Self-rated general well-being.

### Example 5

### ReFerm Significantly Reduces Gut Permeability and Improves Transepithelial Resistance (TER) in Persons With IBS and in a Caco-2 Cell Model

Increased intestinal permeability and microbial dysbiosis are important pathophysiological mechanisms underlying irritable bowel syndrome (IBS). The increased permeability ca be described as leaky gut, which results in the influx from the intestine of large molecules and or microorganisms through the gut barrier and into the body i.e. into the tissues on the body side of the intestine and the blood stream.

The presence of foreign large molecules and microorganisms in the complex gut mucosa gut epithelia area induces an inflammatory reaction. This intestinal inflammation is intense in diseases like ulcerative colitis (Gajendran et al. 2019 Dis Mon 65(12):100851) and so-called low grade in syndromes like IBS (Hirotada Akiho et al 2010 World J Gastrointest Pathophysiol 1(3): 97-105).

Inflammation will also occur in organs like the liver when the foreign large molecules and microorganisms enter the portal vein that carries blood from the gastrointestinal tract to the liver.

On this background, a single blinded randomized clinical trial was conducted with ReFerm on IBS persons with moderate to severe diarrhea (IBS-D) or IBS with mixed diarrhea and constipation (IBD-M). The aim was to analyze whether an intervention with ReFerm would reduce gut permeability in the IBS persons compared to intervention with the placebo product Thick-it®. The placebo product contains artesian mineral water and ≤2% xanthan gum, calcium chloride, malic acid, potassium benzoate, potassium sorbate (to preserve freshness), sodium hexametaphosphate, and disodium ethylenediaminetetraacetic acid (EDTA). The placebo product was chosen because no effect was expected from the product and since it was ideal to mimic the viscosity of ReFerm.

30 persons with IBS were included in the trial and randomized to either ReFerm (18 persons, two men) or Thick-it® (12 persons, three men). There were no significant differences between the groups in terms of age, BMI, or disease severity. The intervention was performed as enema with either 250 mL liquid ReFerm or 250 ml Thick-it® twice daily for 14 days. The reason for choosing this route of administration of the trial products was to apply the products directly in the colon where the gut barrier was expected to be impaired. Sigmoidoscopy was performed twice for each participant at baseline and after 14 days of enema treatment. Colonic biopsies were obtained during the sigmoidoscopy for analysis of gut permeability and TER using an Ussing chamber.

To assess clinical improvement of symptoms, questionnaires were completed twice: before and after the intervention. During the 14 days of the intervention, the participants completed daily questionnaires. However, clinical improvement was not expected since enema twice daily usually will negatively influence the impression of well-being. To improve compliance with the study intervention, the participants were given a check-up call by a principal investigator twice per week during the intervention period.

Four (no men) persons and two (one man) persons dropped out of the ReFerm and placebo arms, respectively.

The methods for analyzing paracellular and transcellular passage and TER in the biopsies and for analyzing the same parameters in the Caco-2 cell model are described in detail in Bednarska et al 2022 Front Nutr 9:1004084. For the in vitro experiments (Caco-2 cell model), 250 ml of ReFerm packages were heat-treated at 80° C., and the liquid ReFerm inside the package was kept at 80° C. for 90 min, followed by cooling to 20° C. The CFU in heat-treated ReFerm was confirmed to be <1 per mL.

The results of this trial (described in detail in Bednarska et al. 2022) were as follows. ReFerm reduced paracellular permeability (p<0.05) and increased transepithelial resistance (TER) over time (p<0.01), whereas the placebo had no significant effect in IBS persons. In ReFerm-treated Caco-2 cells, paracellular and transcellular permeabilities were decreased compared to the control (p<0.05) and placebo (p<0.01). TER was increased in Caco-2 ReFerm treated cells, and normalized TER was increased in ReFerm-treated Caco-2 cells compared to control (p<0.05) and placebo-treated (p<0.05) cells.

The conclusion is that ReFerm significantly reduces paracellular permeability and improves TER in colonic biopsies collected from persons with IBS and in a Caco-2 cell model. This means that ReFerm strengthens the intestinal barrier in IBS persons, which is a prerequisite for achieving a longer-lasting treatment effect for IBS.

As mentioned in the above reference of Gajendran et al., low-grade inflammation plays a pivotal role in the gastrointestinal dysfunction in IBS. Therefore, it will be interesting to analyze whether there is a reduced inflammatory level in the IBS biopsies with improved gut barrier after ReFerm intervention. It will explain the high treatment efficiency if ReFerm both strengthen the gut barrier and reduces intestinal inflammation.

### Example 6

### Effect of ReFerm® on Liver Fibrosis in Alcohol-Related Liver Disease

In a randomized controlled trial, the efficacy and safety of ReFerm was compared to Fresubin (standard nutritional supplement) with alcohol-related liver disease. Patients were allocated in 1:1 ratio to ReFerm® or Fresubin®. The trial was registered on ClinicalTrials.gov Identifier: NCT03863730. Compensated advanced chronic alcohol-related liver disease ages 30 and 75 years old were considered eligible for the trial.

Referm was produced essentially as described in Krag et al. (2012) and Krag et al. (2013).

### Inclusion Criteria

1. Compensated advanced chronic alcohol-related liver disease defined as liver stiffness≥15 kPa (by transient elastography) or a newly performed (<6 mdr) liver biopsy with Kleiner Fibrosis Stage (PMID: 15915461)≥3 or a liver biopsy>6 months with Kleiner Fibrosis Stage≥3 and a current liver stiffness≥10 kPa.
2. A prior or ongoing harmful alcohol intake defined as an average of ≥24 g alcohol/day for women and ≥36 g/d for men for ≥5 years.
3. Informed consent

### Exclusion Criteria

1. Child-Pugh C score and Meld-Na≥15.
2. Hospitalization within three months,
3. Moderate or severe ascites,
4. High-risk varices needing interventional treatment,
5. Other causes of liver disease including viral hepatitis, autoimmune hepatitis, hemochromatosis, Wilson's disease, alfa-1 antitrypsin deficiency, drug-induced liver disease.
6. Antibiotic treatment in the prior three months,
7. Treatment with nutritional drinks, probiotics or prebiotics within the last three months,
8. Lactose intolerance,
9. Coeliac disease,
10. Irritable bowel syndrome defined by ROME III criteria²,
11. Investigator judged that the patient would not be compliant with the trial product,
12. Severe malnutrition,
13. Infectious gastroenteritis in the past six weeks,
14. Pregnancy
15. Malignancy within the past year except planocellular skin cancer and.

### Randomization

Participants were randomized 1:1 to the ReFerm or Fresubin groups. Randomization was performed in variable blocks and stratified according to drinking status at inclusion.

### Intervention

For 24 weeks, each patient orally consumed 250 ml of ReFerm (called one dose) twice daily; one dose as the first meal in the morning approximately one hour before the next meal. The second daily dose was to be taken in the late afternoon at least approximately one hour after the previous meal and at least approximately one hour before the next meal.

One dose of ReFerm contains approximately 35 grams dry weight of oatmeal microbially fermented by L. plantarum 299v, as described by Krag et al. (2012) and Krag et al. (2013), with the addition of 0.1 grams of thiamine per liter ReFerm. Lp299v colony forming units (CFU) in ReFerm ranged from 1.0E+06 to 1.0E+09 per mL.

### Primary Endpoint

A between treatment group comparison of the proportion of patients with a ≥10% reduction of α-SMA in liver biopsies collected at baseline and after 24 weeks of treatment.

### Secondary Endpoints

1. Between group differences in improvement from baseline to 24 weeks in biomarkers indicating severity of a fibrotic liver disease (liver fibrosis)
   a. α-smooth muscle actin (α-SMA).
      i. α-SMA expression is a reliable marker of hepatic stellate cells activation which precedes fibrous tissue deposition (PMID: 15843085)
   b. Liver stiffness measurement (LSM) assessed by transient elastography (TE)
      i. Liver stiffness measured (LSM) by transient elastography is the most widely used and validated technique to assess liver fibrosis in ALD and NAFLD. (PMID: 34166721)
   c. PRO-C3
      i. PRO-C3 is a measurement of the N-terminal pro-peptide of type III collagen and the PRO-C3 collagen neo-epitope is a putative direct marker of fibrogenesis in liver fibrosis in ALD and NAFLD (PMID: 30014517 & PMID: 34251031)
   d. Enhanced liver fibrosis test (ELF test)
      i. The ELF Test is a non-invasive blood test that measures three direct markers of liver fibrosis in ALD and NAFLD (PMID: 29317276 & PMID: 32275982)
2. Between group differences in improvement from baseline to 24 weeks in liver fibrosis according to histological assessment of liver biopsies according to Kleiner fibrosis score (PMID: 15915461).
3. Between group differences in improvement from baseline to 24 weeks in biomarkers indicating disease activity (steatohepatitis).
4. Between group differences in improvement from baseline to 24 weeks in histological features (hepatocyte ballooning, hepatic lobular inflammation, hepatic steatosis) defining disease activity (steatohepatitis) according to the NASH CRN scoring system (PMID: 15915461).

### Results

From March 2018 until January 2021, 56 patients consented to participate were randomly allocated to either ReFerm (n=28) or Fresubin (n=28).

The median age was 63 years, 47 (84%) were males and 28 (50%) reported to be abstinent from alcohol at inclusion for at least the past week prior to enrolment. According to the Kleiner Fibrosis Score four (8%) patients had moderate liver fibrosis (F2), 15 (28%) had severe fibrosis (F3) and 33 (62%) had cirrhosis (F4). The median liver stiffness was 21.1 kPa. In each group, 23 (82%) had the metabolic syndrome.

Forty-one (73%) completed the trial according to protocol with dual liver biopsies meeting quality requirements.

### Effect on Liver Fibrosis

### Effect on α-SMA

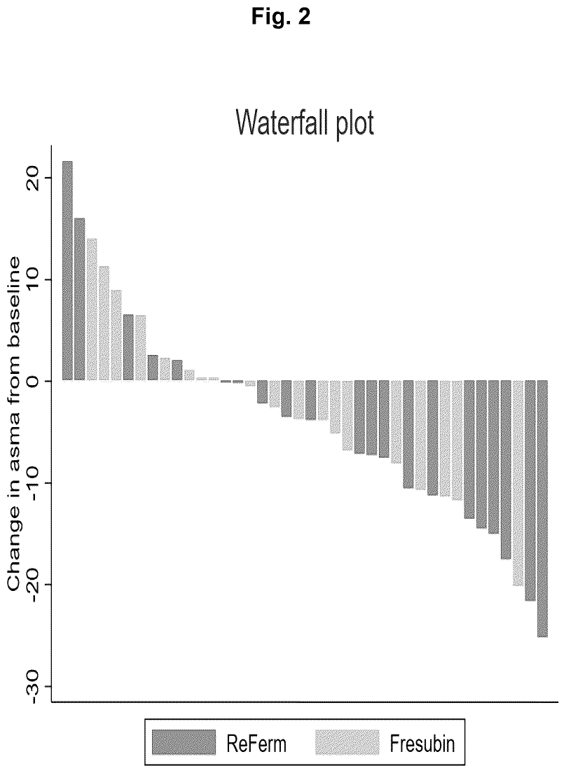

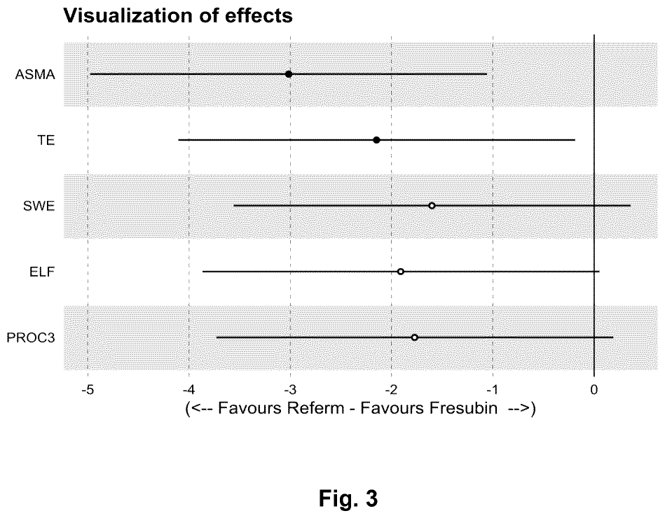

Result 1: In the per protocol analysis ReFerm reduced α-SMA expression by at least 10% more frequently compared to Fresubin (8/21 in the ReFerm group vs 4/20 in the Fresubin group; OR=0.43, 95% confidence interval [CI], 0.10 to 1.79; P=0.246) FIG. 2. The results were consistent in the ITT analysis (OR 0.42, 95% CI; 0.11 to 1.59; P=0.20).
Result 2: After treatment the Referm group had reduced α-SMA expression by −5.32% (95% CI; −10.55 to −0.09) and Fresubin had reduced α-SMA expression by −2.05% (95% CI; −6.20 to 2.10). The mean difference in change was −3.27% (95% CI −9.82 to 3.29, p=0.320). Table 1
Result 3: In the adjusted model Referm significantly reduced α-SMA expression (FIG. 3). In the model the effect was adjusted for age, sex, alcohol consumption and magnitude of fibrosis at baseline.

### Effect on Liver Stiffness

Result 4: ReFerm reduced LSM by at least 5 kPa more frequently compared to Fresubin (9/21 in the ReFerm group vs 1/20 in the Fresubin group; OR=0.07, 95% confidence interval [CI], 0.01 to 0.63; P=0.017)
Result 5: ReFerm reduced LSM by at least 20% more frequently compared to Fresubin (8/21 in the ReFerm group vs 2/20 in the Fresubin group; OR=0.18, 95% CI, 0.03 to 0.99; P=0.049)
Result 6: Referm reduced LSM with a mean of −4.5 kPa (95% CI, −10.9 to 1.9) and LSM increased by +0.8 kPa (−3.2 to 4.8) in the Fresubin group after treatment. The mean difference in change was −5.3 kPa (95% CI, −12.6 to 2.1, P=0.158). Table 1
Result 7: In the adjusted model Referm significantly reduced LSM (TE) (FIG. 3). In the model the effect was adjusted for age, sex, alcohol consumption and magnitude of fibrosis at baseline.

### Effect on PRO-C3

Result 8: Referm reduced PRO-C3 with a mean of −1.91 ng/L (95% CI, −4.38 to 0.56) and PRO-C3 increased by +1.60 ng/L (0.31 to 4.50) in the Fresubin group after treatment. The mean difference in change was −3.51 ng/L (95% CI, −7.19 to 0.18, P=0.062). Table 1
Result 9: In the adjusted model Referm significantly reduced PRO-C3 (FIG. 3). In the model the effect was adjusted for age, sex, alcohol consumption and magnitude of fibrosis at baseline.

### Effect on ELF

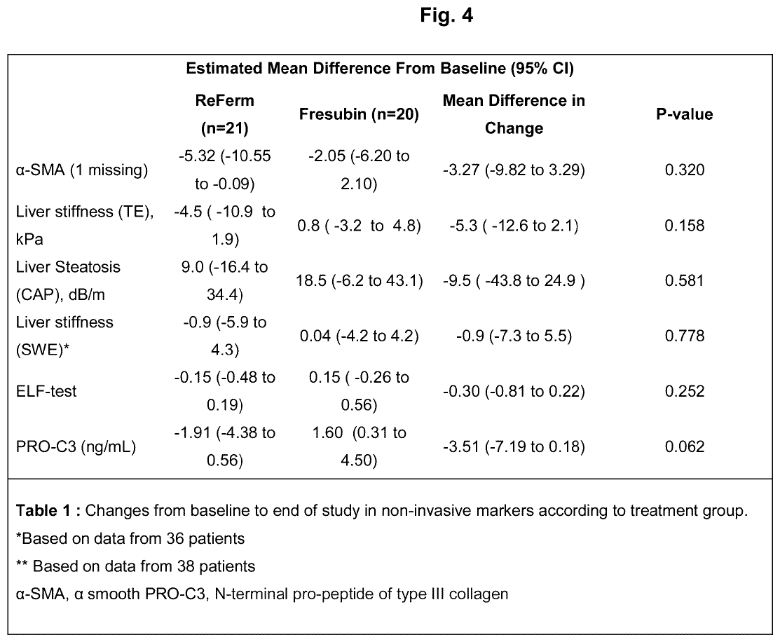

Result 10: Referm reduced ELF with a mean of −0.15 (95% CI, −0.48 to 0.19) and ELF increased by +0.15 (−0.26 to 0.56) in the Fresubin group after treatment. The mean difference in change was −0.30 (95% CI, −0.81 to 0.22, P=0.252). See Table 1 in FIG. 4.
Result 11: In the adjusted model Referm significantly reduced ELF (FIG. 3). In the model the effect was adjusted for age, sex, alcohol consumption and magnitude of fibrosis at baseline.

### Effect on Histology

ReFerm reduced liver fibrosis by at least 1 stage according to the Kleiner Fibrosis score more frequently compared to Fresubin (6/21 in the ReFerm group vs 3/20 in the Fresubin group; OR=0.44, 95% confidence interval [CI], 0.09 to 2.08; P=0.301)

### Effect on Steatohepatitis

### Effect on Histology

Referm reduced worsening of hepatic lobular inflammation compared to Fresubin (1/21 in the ReFerm group and 5/20 in the Fresubin group; OR=0.15, 95% CI; 0.02 to 1.42 (p=0.098).

### Effect on Hepatic Steatosis

### Effect on Controlled Attenuation Parameter (CAP)

Referm group had a lower increase of hepatic steatosis 9 (−16 to 34) dB/m compared the to Fresubin group 19 dB/m (−6 to 43) measured by controlled attenuation parameter. The mean difference in change was −10 dB/m (95% CI, −44 to 25, P=0.581)

### Generalizability to NAFLD

ALD and NAFLD share many features and from a clinical perspective the only way to distinguish is the history of alcohol consumption. Traditionally ALD is characterized by a history of a high alcohol consumption (PMID: 29628280), while NAFLD is characterized by low alcohol consumption and is usually seen in people with metabolic risk factors such as overweight or with obesity, diabetes, high blood pressure and dyslipidemia (PMID: 27062661). However, the many people with an excessive alcohol use also have these metabolic risk factors (PMID: 33279778), and people with metabolic risk factors and just a moderate alcohol use are in higher risk of developing fatty liver disease and fibrotic liver disease (PMID: 34606913).

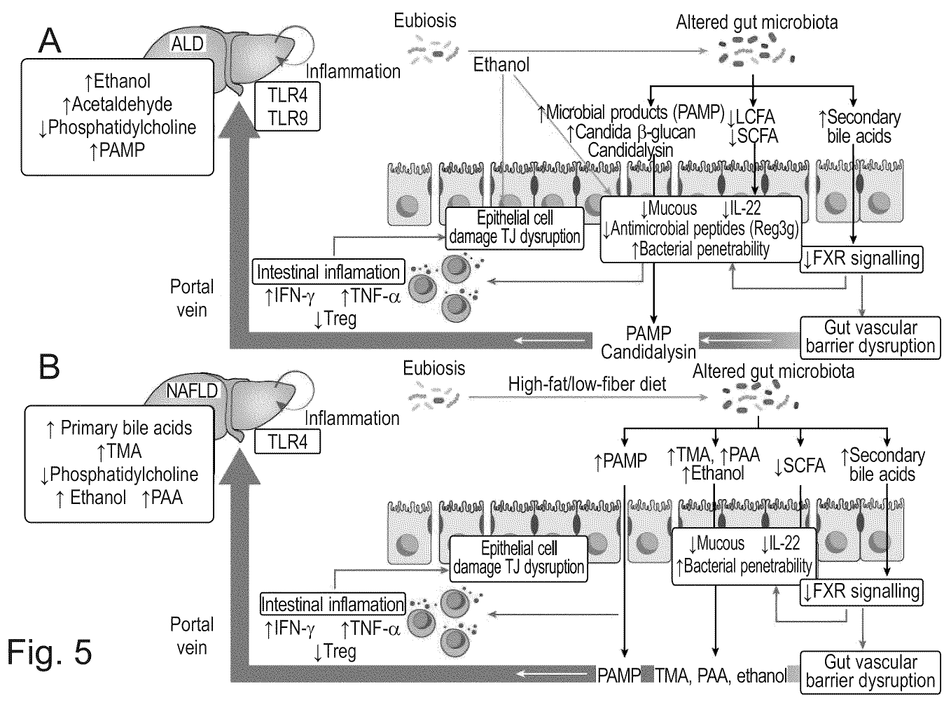

From a pathophysiological point of view ALD and NAFLD share many common features comprised by the gut-liver axis (FIG. 5): Altered gut microbiome, impaired gut permeability, translocation of microbial product from the gut to the liver and intestinal inflammation that all lead to hepatic inflammation (steatohepatitis) which activates fibrogenesis (development and progression of fibrotic liver disease) (PMID: 31622696). It is well established that interventions targeting the gut-liver axis are potential therapies for both ALD and NAFLD (PMID: 28526488. & PMID: 31622696).

This study illustrates that ReFerm can improve liver fibrosis, steatohepatitis and hepatic steatosis. Although the mode of action of ReFerm has not yet been clarified, it is very likely that the beneficial effects of ReFerm are mediated by improvements in the gut-liver axis. It is therefore likely that ReFerm can also improve liver fibrosis, steatohepatitis and hepatic steatosis people with NAFLD regardless of alcohol consumption.

### Example 7

### Assessment of the Mode of Action of Fermented Cereal Compositions in Liver Diseases

Bednarska et al. 2022 (ibid.) demonstrates that fermented cereal compositions obtained by fermenting a cereal gruel with L. plantarum 299v provide a significant improvement of the gut barrier function in persons with irritable bowel syndrome (IBS).

A deficient gut barrier may be a result, e.g., of dysbiosis of the gut floraor a reduced gut barrier function that results in transport or translocation of undesirable molecules and/or microorganism from the gut to the liver. It is believed that this is a likely reason for the onset and/or progression of liver fibrosis and cirrhosis (Schwenger et al. 2019 JHEP Rep. 1(3): pp. 214-226).

Patients suffering from liver fibrosis obtain an improved liver status when administered ReFerm on a daily basis. The improved liver status is believed to be obtained because of a significant improvement of the permeability of the gut barrier.

There are at least two analytical methods available for testing this suggestion.

Blood samples from patients fulfilling inclusion criteria may be analyzed for the amount of marker molecules, such as, e.g., Zonulin, and Lipopolysaccharide (LPS). The relative amount of marker molecules is correlated to mucosal or gut barrier injury. In addition to Zonulin and LPS, a marker molecule such as, e.g., Intestinal Fatty Acid Binding Protein (I-FABP; FABP2) may be used as a marker for mucosal or gut barrier injury (Schoultz and Keita (2020), Cells 9(8): p. 1909).

Colon biopsies on liver patients may be conducted before and after intervention with ReFerm and placebo. The biopsy tissues are analyzed for barrier properties in an Ussing chamber. This method is among the most advanced methods used today (Schoultz and Keita (2020), Cells 9(8): 1909).

ReFerm improves the gut barrier in liver fibrosis/cirrhosis, if the gut barrier is significantly improved in the ReFerm arm vs the placebo arm and/or in the group with significant improved liver status vs no significant liver status in the ReFerm arm.

1. Fermented cereal composition obtained by fermenting a cereal gruel with one or more microorganisms selected from Lactobacillus plantarum 299v and Lactobacillus plantarum 299 for use in treatment of a fatty liver disease or a fibrotic liver disease in an individual.

2. The fermented cereal composition according to claim 1, wherein a daily administration of an efficient amount of the fermented cereal composition reduces disease symptoms of the individual or normalizes disease markers in the individual.

3. The fermented cereal composition according to any of claims 1 and 2, wherein the treatment prevents or postpones progression or initiates regression of the liver disease.

4. The fermented cereal composition according to any of claims 1 to 3, wherein the liver disease is a non-alcoholic fatty liver disease, including steatosis or steatohepatitis.

5. The fermented cereal composition according to any of claims 1 to 3, wherein the liver disease is an alcoholic liver disease, including alcoholic steatosis and alcoholic steatohepatitis.

6. The fermented cereal composition according to any of claims 1 to 3, wherein the fibrotic liver disease is alcoholic liver fibrosis or alcoholic cirrhosis.

7. The fermented cereal composition according to any of claims 1 to 3, wherein the fibrotic liver disease is fibrotic steatosis, fibrotic steatohepatitis or cirrhosis.

8. The fermented cereal composition according to any of claims 1 to 7, wherein the fatty liver disease or the fibrotic liver disease is associated with gut-dysbiosis.

9. The fermented cereal composition according to any of claims 1 to 8, wherein the fermented cereal composition has a pH below 5.5 when the fermentation is completed.

10. The fermented cereal composition according to any of claims 1 to 9, wherein some of the microorganisms in the fermented cereal composition are alive.

11. The fermented cereal composition according to any of claims 1 to 9, wherein some of the microorganisms in the fermented cereal composition are dead.

12. The fermented cereal composition according to any of claims 1 to 9, wherein the fermented cereal composition is a dried preparation.

13. The fermented cereal composition according to claim 12, wherein the dried preparation is a freeze dried or spray dried preparation.

14. The fermented cereal composition according to any of claims 12 and 13, wherein some of the microorganisms in the dried preparation of the fermented cereal composition are dead.

15. The fermented cereal composition according to any of claims 1 to 14, wherein probiotic microorganisms are added to the fermented cereal composition.

16. A combination of an efficient amount of a fermented cereal composition and an efficient amount of one or both of Lactobacillus plantarum 299v and Lactobacillus plantarum 299 for use in the treatment of a fatty liver disease or a fibrotic liver disease in an individual.

17. The combination according to claim 16, wherein a daily administration of the combination reduces disease symptoms of the individual or normalizes disease markers in the individual.

18. The combination according to any of claims 16 and 17, wherein the treatment prevents or postpones progression or initiates regression of the liver disease.

19. The combination according to any of claims 16 to 18, wherein the liver disease is a non-alcoholic fatty liver disease, including steatosis or steatohepatitis.

20. The combination according to any of claims 16 to 18, wherein the liver disease is an alcoholic liver disease, including alcoholic steatosis and alcoholic steatohepatitis.

21. The combination according to any of claims 16 to 18, wherein the fibrotic liver disease is alcoholic liver fibrosis or alcoholic cirrhosis.

22. A dosage form suitable for daily intake of a cereal gruel fermented with one or both of Lactobacillus plantarum 299v and Lactobacillus plantarum 299 for use in the treatment of a fatty liver disease or a fibrotic liver disease in an individual.

23. The dosage form according to claim 22, wherein a daily administration of the combination reduces disease symptoms of the individual or normalizes disease markers in the individual.

24. The dosage form according to any of claims 22 and 23, wherein the treatment prevents or postpones progression or initiates regression of the liver disease.

25. The dosage form according to any of claims 22 to 24, wherein the liver disease is a non-alcoholic fatty liver disease, including steatosis or steatohepatitis.

26. The dosage form according to any of claims 22 to 24, wherein the liver disease is an alcoholic liver disease, including alcoholic steatosis and alcoholic steatohepatitis.

27. The dosage form to any of claims 22 to 24, wherein the fibrotic liver disease is alcoholic liver fibrosis or alcoholic cirrhosis.
